(19) United States
(12) Patent Application Publication
(10) Pub. No.: US 2018/0325943 A1
(43) Pub. Date: Nov. 15, 2018

(54) Biocompatible Articles With Embedded Copper Ions and Copper Ion Releasing Coating

(71) Applicant: Curical Technologies Ltd., Herzliya (IL)

(72) Inventors: Inbal Loitner, Rishon Le Zion (IL); Shai Garty, Rehovot (IL); Dafna Gabbay, Tel Aviv (IL)

(73) Assignee: Curical Technologies Ltd., Herzliya (IL)

(21) Appl. No.: 15/775,055

(22) PCT Filed: Nov. 11, 2016
(86) PCT No.: PCT/IL2016/051221

§ 371 Date: May 10, 2018

Related U.S. Application Data
(60) Provisional application No. 62/254,395, filed on Nov. 12, 2015.
(60) Provisional application No. 62/254,398, filed on Nov. 12, 2015.
(60) Provisional application No. 62/254,414, filed on Nov. 12, 2015.

(51) Int. Cl. A61K 33/34; A61L 17/00; A61L 17/14; A61L 24/00; A61L 24/06; A61L 27/16

(52) U.S. Cl. CPC A61K 33/34; A61L 17/005; A61L 17/145; A61L 24/0015; A61L 2430/02; A61L 27/16; A61L 2300/102; A61L 2300/404; A61L 24/06

(57) ABSTRACT

A suture including at least one filament formed of at least one polymer and at least one copper ion at least partially embedded in the at least one filament in a manner such that the at least one copper ion is released from the at least one filament over time.

### REFERENCE TO RELATED APPLICATIONS

Reference is hereby made to U.S. Provisional Patent Application Ser. No. 62/254,395, filed Nov. 12, 2015 and entitled SUTURE WITH EMBEDDED COPPER IONS, U.S. Provisional Patent Application Ser. No. 62/254,398, filed Nov. 12, 2015 and entitled BIOCOMPATIBLE ARTICLES WITH COPPER ION RELEASING COATING and U.S. Provisional Patent Application Ser. No. 62/254,414, filed Nov. 12, 2015 and entitled BIOCOMPATIBLE ARTICLES WITH EMBEDDED COPPER IONS AND COPPER ION RELEASING COATING, the disclosures of which are hereby incorporated by reference and priority of which are hereby claimed pursuant to 37 C.F.R. 1.78(a)(1).

### FIELD OF THE INVENTION

The present invention relates to biocompatible articles generally and more particularly to biocompatible articles including at least one copper ion.

### BACKGROUND OF THE INVENTION

Various biocompatible articles including at least one copper ion are known.

### SUMMARY OF THE INVENTION

The present invention seeks to provide improved biocompatible articles having at least one copper ion at least partially embedded therein and/or at least one copper ion at least partially coated thereon.

There is thus provided in accordance with a preferred embodiment of the present invention a suture including at least one filament formed of at least one polymer and a biodegradable coating including at least one copper ion at least partially coated on the at least one filament in a manner such that the at least one copper ion is released from the biodegradable coating over time.

There is also provided in accordance with another preferred embodiment of the present invention a suture including at least one filament and a biodegradable coating including at least one copper ion at least partially coated on the at least one filament in a manner such that the at least one copper ion is released from the biodegradable coating over time.

Preferably, the biodegradable coating includes at least one biodegradable polymer. In accordance with a preferred embodiment of the present invention the biodegradable coating includes at least one aliphatic polyester. Additionally, the at least one aliphatic polyester is selected from the group consisting of: polymers polymerized from one or more of the following: ε-caprolactone, lactide, glycolide, dioxanone and copolymers thereof. In accordance with a preferred embodiment of the present invention the at least one aliphatic polyester includes PLGA.

In accordance with a preferred embodiment of the present invention the biodegradable coating includes a copolymer made from 65% D,L-lactide and 35% glycolide.

Preferably, the biodegradable coating includes a copper chloride solution. Preferably, the copper chloride solution is a 1-2% wt./wt. solution. In accordance with a preferred embodiment of the present invention the copper chloride solution is a 2% wt./wt. solution. In accordance with a preferred embodiment of the present invention the copper chloride solution is a 5% wt./wt. solution. In accordance with a preferred embodiment of the present invention the copper chloride solution is a 1-5% wt./wt. solution. In accordance with a preferred embodiment of the present invention the copper chloride solution is a 1-10% wt./wt. solution.

In accordance with a preferred embodiment of the present invention the biodegradable coating includes a copper sulfate solution. In accordance with a preferred embodiment of the present invention the copper sulfate solution is a 1-2% wt./wt. solution. In accordance with a preferred embodiment of the present invention the copper sulfate solution is a 2% wt./wt. solution. In accordance with a preferred embodiment of the present invention the copper sulfate solution is a 5% wt./wt. solution. In accordance with a preferred embodiment of the present invention the copper sulfate solution is a 1-5% wt./wt. solution. In accordance with a preferred embodiment of the present invention the copper sulfate solution is a 1-10% wt./wt. solution.

Preferably, the biodegradable coating also includes at least one lubricant. Additionally, the at least one lubricant is selected from the group consisting of copper stearate and calcium stearate.

In accordance with a preferred embodiment of the present invention the at least one filament is biodegradable. Preferably, the at least one filament includes at least one aliphatic polyester. Additionally, the at least one aliphatic polyester is selected from the group consisting of: polymers polymerized from one or more of the following: ε-caprolactone, lactide, glycolide, dioxanone and copolymers thereof.

In accordance with a preferred embodiment of the present invention the at least one filament includes a copolymer made from 90% glycolide and 10% L-lactide.

There is further provided in accordance with yet another preferred embodiment of the present invention a biocompatible article including at least one element formed of at least one polymer and a biodegradable coating including at least one copper ion at least partially coated on the at least one element in a manner such that the at least one copper ion is released from the biodegradable coating over time.

There is still further provided in accordance with still another preferred embodiment of the present invention a biocompatible article including at least one element and a biodegradable coating including at least one copper ion at least partially coated on the at least one element in a manner such that the at least one copper ion is released from the biodegradable coating over time.

Preferably, the biodegradable coating includes at least one biodegradable polymer. In accordance with a preferred embodiment of the present invention the biodegradable coating includes at least one aliphatic polyester. Additionally, the at least one aliphatic polyester is selected from the group consisting of: polymers polymerized from one or more of the following: ε-caprolactone, lactide, glycolide, dioxanone and copolymers thereof. In accordance with a preferred embodiment of the present invention the at least one aliphatic polyester includes PLGA.

In accordance with a preferred embodiment of the present invention the biodegradable coating includes a copolymer made from 65% D,L-lactide and 35% glycolide.

Preferably, the at least one element is biodegradable.

In accordance with a preferred embodiment of the present invention the biocompatible article is selected from a suture, a mesh tissue management device, a wound closure device and a tissue engineering device.

There is even further provided in accordance with another preferred embodiment of the present invention a suture including at least one filament formed of at least one polymer and at least one copper ion at least partially embedded in the at least one filament in a manner such that the at least one copper ion is released from the at least one filament over time.

In accordance with a preferred embodiment of the present invention the at least one filament includes at least one of a monofilament and a multifilament.

Preferably, the at least one polymer includes at least one biodegradable polymer. In accordance with a preferred embodiment of the present invention the at least one biodegradable polymer includes at least one aliphatic polyester. Additionally, the at least one aliphatic polyester is selected from the group consisting of: polymers polymerized from one or more of the following: ε-caprolactone, lactide, glycolide, dioxanone and copolymers thereof. In accordance with a preferred embodiment of the present invention the at least one aliphatic polyester includes PLGA.

In accordance with a preferred embodiment of the present invention the at least one aliphatic polyester includes a copolymer made from 90% glycolide and 10% L-lactide.

Preferably, the at least one copper ion is provided by at least one of copper chloride (CuCl₂) and copper sulfate (CuSO₄). Alternatively, the at least one copper ion is provided by copper oxide (Cu₂O).

In accordance with a preferred embodiment of the present invention the suture also includes at least one plasticizer selected from the group consisting of: Aliphatic short oligomers of Homopolymer such as Polyglycolide or Polyglycolic acid (PGA) Polylactic acid (PLA), Polycaprolactone (PCL), Polyhydroxyalkanoate (PHA), Polyhydroxybutyrate (PHB) or copolymer thereof, or copolymers of Polyethylene adipate (PEA), Polybutylene succinate (PBS). Additionally or alternatively, the suture also includes at least one of a short oligomer of poly(ε-caprolactone) and a short oligomer of poly(ethylene glycol).

Preferably, the suture also includes an antioxidant. Additionally, the antioxidant is Tris(nonylphenyl) phosphate.

In accordance with a preferred embodiment of the present invention the suture also includes a metal deactivator additive of 2′, 3-bis [[3-[3, 5-di-tert-butyl-4-hydroxyphenyl] propionyl]] propionohydrazide.

There is also provided in accordance with yet another preferred embodiment of the present invention a biocompatible article including at least one element formed of at least one biodegradable polymer and at least one copper ion at least partially embedded in the at least one element in a manner such that the at least one copper ion is released from the at least one element over time.

Preferably, the at least one copper ion is provided by at least one of copper chloride (CuCl₂), copper sulfate (CuSO₄), and copper oxide (Cu₂O).

There is further provided in accordance with still another preferred embodiment of the present invention a biocompatible article including at least one element formed of at least one polymer and at least one copper ion, selected from copper sulfate and copper chloride, at least partially embedded in the at least one element in a manner such that the at least one copper ion is released from the at least one element over time.

Preferably, the at least one biodegradable polymer includes at least one aliphatic polyester. Additionally, the at least one aliphatic polyester is selected from the group consisting of: polymers polymerized from one or more of the following: ε-caprolactone, lactide, glycolide, dioxanone and copolymers thereof. In accordance with a preferred embodiment of the present invention the at least one aliphatic polyester includes PLGA. In accordance with a preferred embodiment of the present invention the at least one aliphatic polyester includes a copolymer made from 90% glycolide and 10% L-lactide.

In accordance with a preferred embodiment of the present invention the biocompatible article also includes at least one plasticizer selected from the group consisting of: Aliphatic short oligomers of Homopolymer such as Polyglycolide or Polyglycolic acid (PGA) Polylactic acid (PLA), Polycaprolactone (PCL), Polyhydroxyalkanoate (PHA), Polyhydroxybutyrate (PHB) or copolymer thereof, or copolymers of Polyethylene adipate (PEA), Polybutylene succinate (PBS). Additionally or alternatively, the biocompatible article also includes at least one of a short oligomer of poly(ε-caprolactone) and a short oligomer of poly(ethylene glycol).

In accordance with a preferred embodiment of the present invention the biocompatible article also includes an antioxidant. Additionally, the antioxidant is Tris(nonylphenyl) phosphate.

Preferably, the biocompatible article also includes a metal deactivator additive of 2′, 3-bis [[3-[3, 5-di-tert-butyl-4-hydroxyphenyl] propionyl]] propionohydrazide.

In accordance with a preferred embodiment of the present invention the biocompatible article is implantable.

There is still further provided in accordance with yet another preferred embodiment of the present invention a surgical glue including at least one gluing agent and at least one copper ion, at least partially embedded in the at least one gluing agent in a manner such that the at least one copper ion is released from the at least one gluing agent over time.

There is yet further provided in accordance with another preferred embodiment of the present invention a bone cement including at least one acrylic powder and at least one copper ion, at least partially embedded in the at least one acrylic powder in a manner such that the at least one copper ion is released from the at least one acrylic powder over time.

There is also provided in accordance with yet another preferred embodiment of the present invention a suture including at least one filament, at least one first copper ion at least partially embedded in the at least one filament in a manner such that the at least one first copper ion is released from the at least one filament over time and a biodegradable coating including at least one second copper ion at least partially coated on the at least one filament in a manner such that the at least one second copper ion is released from the biodegradable coating over time.

In accordance with a preferred embodiment of the present invention the at least one filament includes at least one of a monofilament and a multifilament.

Preferably, the at least one filament is formed of at least one polymer. Additionally, the at least one polymer includes at least one biodegradable polymer.

In accordance with a preferred embodiment of the present invention the at least one biodegradable polymer includes at least one aliphatic polyester. Additionally, the at least one aliphatic polyester is selected from the group consisting of: polymers polymerized from one or more of the following: ε-caprolactone, lactide, glycolide, dioxanone and copolymers thereof. In accordance with a preferred embodiment of the present invention the at least one aliphatic polyester includes PLGA. In accordance with a preferred embodiment of the present invention the at least one aliphatic polyester includes a copolymer made from 90% glycolide and 10% L-lactide.

Preferably, the biodegradable coating includes at least one biodegradable polymer. Additionally, the at least one biodegradable polymer includes at least one aliphatic polyester. In accordance with a preferred embodiment of the present invention the at least one aliphatic polyester is selected from the group consisting of: polymers polymerized from one or more of the following: ε-caprolactone, lactide, glycolide, dioxanone and copolymers thereof. Additionally, the at least one aliphatic polyester includes PLGA. In accordance with a preferred embodiment of the present invention the at least one aliphatic polyester includes a copolymer made from 65% D,L-lactide and 35% glycolide

Preferably, the at least one first copper ion is provided by at least one of copper chloride (CuCl₂), copper sulfate (CuSO₄), and copper oxide (Cu₂O). Additionally or alternatively, the at least one second copper ion is provided by at least one of copper chloride (CuCl₂), copper sulfate (CuSO₄), and copper oxide (Cu₂O).

There is still further provided in accordance with still another preferred embodiment of the present invention a biocompatible article including at least one element, at least one first copper ion at least partially embedded in the at least one element in a manner such that the at least one first copper ion is released from the at least one element over time and a biodegradable coating including at least one second copper ion at least partially coated on the at least one element in a manner such that the at least one second copper ion is released from the biodegradable coating over time.

Preferably, the at least one element is formed of at least one polymer. Additionally, the at least one polymer includes at least one biodegradable polymer.

In accordance with a preferred embodiment of the present invention the biocompatible article is implantable.

In accordance with a preferred embodiment of the present invention the biocompatible article is selected from a suture, a mesh tissue management device, a wound closure device and a tissue engineering device.

There is yet further provided in accordance with another preferred embodiment of the present invention a method of manufacture of a suture, the method including forming at least one filament of at least one polymer and at least partially coating the at least one filament with a biodegradable coating including at least one copper ion in a manner such that the at least one copper ion is released from the biodegradable coating over time.

There is even further provided in accordance with still another preferred embodiment of the present invention a method of manufacture of a suture, the method including forming at least one filament and at least partially coating the at least one filament with a biodegradable coating including at least one copper ion in a manner such that the at least one copper ion is released from the biodegradable coating over time.

Preferably, the method also includes adding at least one lubricant to at least one of the at least one filament and the biodegradable coating.

There is also provided in accordance with yet another preferred embodiment of the present invention a method of manufacture of a biocompatible article, the method including forming at least one element of at least one polymer and at least partially coating the at least one element with a biodegradable coating including at least one copper ion in a manner such that the at least one copper ion is released from the biodegradable coating over time.

There is further provided in accordance with still another preferred embodiment of the present invention a method of manufacture of a biocompatible article, the method including forming at least one element and at least partially coating the at least one element with a biodegradable coating including at least one copper ion in a manner such that the at least one copper ion is released from the biodegradable coating over time.

Preferably, the method also includes adding at least one lubricant to at least one of the at least one element and the biodegradable coating.

There is yet further provided in accordance with yet another preferred embodiment of the present invention a method of manufacture of a suture, the method including forming at least one filament of at least one polymer and at least partially embedding at least one copper ion in the at least one filament in a manner such that the at least one copper ion is released from the filament over time.

In accordance with a preferred embodiment of the present invention the forming at least one filament includes providing a master batch of polymeric granules including a first concentration of copper, providing pure polymer, forming a polymeric composition by mixing a selected quantity of the polymeric granules including a first concentration of copper with a selected quantity of the pure polymer, the polymeric composition having a second concentration of copper less than the first concentration of copper and forming the at least one filament from the polymeric composition. Additionally, the master batch includes at least one of a plasticizer, an antioxidant and a metal deactivator.

There is still further provided in accordance with still another preferred embodiment of the present invention a method of manufacture of a biocompatible article, the method including forming at least one element of at least one biodegradable polymer and at least partially embedding at least one copper ion in the at least one element in a manner such that the at least one copper ion is released from the element over time.

There is even further provided in accordance with another preferred embodiment of the present invention a method of manufacture of a biocompatible article, the method including forming at least one element of at least one polymer and at least partially embedding at least one copper ion, selected from copper sulfate and copper chloride, in the at least one element in a manner such that the at least one copper ion is released from the element over time.

Preferably, the forming at least one element includes providing a master batch of polymeric granules including a first concentration of copper, providing pure polymer, forming a polymeric composition by mixing a selected quantity of the polymeric granules including a first concentration of copper with a selected quantity of the pure polymer, the polymeric composition having a second concentration of copper less than the first concentration of copper and forming the at least one element from the polymeric composition. Additionally, the master batch includes at least one of a plasticizer, an antioxidant and a metal deactivator.

There is also provided in accordance with yet another preferred embodiment of the present invention a method of manufacture of a surgical glue, the method including providing at least one gluing agent and at least partially embedding at least one copper ion in the at least one gluing agent in a manner such that the at least one copper ion is released from the gluing agent over time.

There is further provided in accordance with still another preferred embodiment of the present invention a method of manufacture of a bone cement, the method including providing at least one acrylic powder and at least partially embedding at least one copper ion in the at least one acrylic powder in a manner such that the at least one copper ion is released from the acrylic powder over time.

There is still further provided in accordance with another preferred embodiment of the present invention a method of manufacture of a suture, the method including forming at least one filament, at least partially embedding at least one first copper ion in the at least one filament in a manner such that the at least one copper ion is released from the filament over time and at least partially coating the at least one filament with a biodegradable coating including at least one second copper ion in a manner such that the at least one copper ion is released from the biodegradable coating over time.

In accordance with a preferred embodiment of the present invention the forming at least one filament includes providing a master batch of polymeric granules including a first concentration of copper, providing pure polymer, forming a polymeric composition by mixing a selected quantity of the polymeric granules including a first concentration of copper with a selected quantity of the pure polymer, the polymeric composition having a second concentration of copper less than the first concentration of copper and forming the at least one filament from the polymeric composition. Additionally, the master batch includes at least one of a plasticizer, an antioxidant and a metal deactivator.

There is yet further provided in accordance with another preferred embodiment of the present invention a method of manufacture of a biocompatible article, the method including forming at least one element, at least partially embedding at least one first copper ion in the at least one element in a manner such that the at least one copper ion is released from the element over time and at least partially coating the at least one filament with a biodegradable coating including at least one second copper ion in a manner such that the at least one copper ion is released from the biodegradable coating over time.

Preferably, the forming at least one element includes providing a master batch of polymeric granules including a first concentration of copper, providing pure polymer, forming a polymeric composition by mixing a selected quantity of the polymeric granules including a first concentration of copper with a selected quantity of the pure polymer, the polymeric composition having a second concentration of copper less than the first concentration of copper and forming the at least one element from the polymeric composition. Additionally, the master batch includes at least one of a plasticizer, an antioxidant and a metal deactivator.

There is still further provided in accordance with still another preferred embodiment of the present invention a master batch of a polymer including copper, the master batch including at least one water soluble copper compound mixed in the polymer, wherein a concentration of the at least one water soluble copper compound in the polymer is between 2% and 40% by weight.

Preferably, the at least one water soluble copper compound includes at least one of Copper chloride (CuCl₂), Copper sulfate (CuSO₄). Additionally or alternatively, the polymer is a biodegradable polymer. In accordance with a preferred embodiment of the present invention the biodegradable polymer is PLGA.

There is also provided in accordance with another preferred embodiment of the present invention a master batch of a polymer including copper, the master batch including a biodegradable polymer and Copper oxide (Cu₂O) mixed in the biodegradable polymer, wherein a concentration of the Copper oxide (Cu₂O) in the biodegradable polymer is between 2% and 40% by weight.

Preferably, the biodegradable polymer is PLGA.

In accordance with a preferred embodiment of the present invention the master batch of a polymer including copper also includes at least one of a plasticizer, an antioxidant and a metal deactivator.

There is further provided in accordance with yet another preferred embodiment of the present invention a method of manufacture of a master batch of a polymer including copper, the method including mixing in the polymer at least one water soluble copper compound, wherein a concentration of the at least one water soluble copper compound in the polymer is between 2% and 40% by weight.

Preferably, the at least one water soluble copper compound includes at least one of Copper chloride (CuCl₂), Copper sulfate (CuSO₄).

There is still further provided in accordance with still another preferred embodiment of the present invention a method of manufacture of a master batch of a biodegradable polymer including copper, the method including mixing Copper oxide (Cu₂O) in the biodegradable polymer, wherein a concentration of the Copper oxide (Cu₂O) in the biodegradable polymer is between 2% and 40% by weight.

Preferably, the method also includes adding at least one of a plasticizer, an antioxidant and a metal deactivator to the polymer.

### BRIEF DESCRIPTION OF THE DRAWINGS

The present invention will be understood and appreciated more fully from the following detailed description, taken in conjunction with the drawings, in which:

FIG. 1 shows a structural diagram of Tris(nonylphenyl) phosphate (TNPP);

FIG. 2 shows a structural diagram of metal deactivator (2′, 3-bis [[3-[3, 5-di-tert-butyl-4-hydroxyphenyl] propionyl]] propionohydrazide);

FIGS. 3A, 3B and 3C are scanning electron microscope (SEM) micrographs of a cross-section of monofilament compounded and embedded fiber of PLGA 10/90 with copper sulfate (PLGA Cu-Sulfate), copper sulfate and PEG (PLGA PEG Cu-Sulfate) and copper sulfate with PCL (PLGA PCL Cu-Sulfate), respectively;

FIG. 4 is a graph showing Differential scanning calorimetry (DSC) analysis of a monofilament, extruded with copper sulfate and TNPP additive;

FIG. 5 is a graph showing a summary of enthalpy for recrystallization, melting and its difference analyzed using DSC of a monofilament, extruded with copper sulfate and additives;

FIGS. 6A, 6B and 6C are SEM micrographs of a cross-section of monofilament compounded and embedded fiber of PLGA 10/90, with copper oxide (PLGA Cu-Oxide), copper oxide and PEG (PLGA PEG Cu-Oxide) and copper oxide with PCL (PLGA PCL Cu-Oxide), respectively;

FIG. 7 is a graph showing Differential scanning calorimetry (DSC) analysis of a monofilament, extruded with copper oxide;

FIG. 8 is a graph showing a summary of enthalpy for recrystallization, melting and its difference analyzed using DSC of a monofilament, extruded with copper oxide;

FIGS. 9A, 9B and 9C are SEM micrographs of a cross-section of monofilament compounded and embedded fiber of PLGA 10/90, with copper chloride (PLGA Cu-chloride), copper chloride and PEG (PLGA PEG Cu-chloride) and copper chloride with PCL (PLGA PCL Cu-chloride), respectively;

FIG. 10 is a graph showing Differential scanning calorimetry (DSC) analysis of a monofilament, extruded with copper chloride with PCL additive;

FIG. 11 is a graph showing a summary of enthalpy for recrystallization, melting and its difference analyzed using DSC of a monofilament, extruded with copper chloride with PCL additive;

FIG. 12 shows a structural diagram of solid organophosphate ULTRANOX 626 phosphite Antioxidant of Bis(2,4-di-tert-butylphenyl) pentaerythritoldiphosphite;

FIGS. 13A and 13B are SEM illustrations, taken at a magnification of ×150, of a suture surface with a coating solution of 5% wt/wt and 10% wt/wt, respectively;

FIGS. 14A, 14B and 14C are SEM illustrations, taken at a magnification of ×150, of a suture surface at a dipping time of 5 sec, 10 sec and 15 sec, respectively;

FIGS. 15A and 15B are SEM illustrations, taken at a magnification of ×150, of a suture surface with a coating including copper chloride and copper sulfate, respectively;

FIGS. 16A and 16B are SEM illustrations, taken at a magnification of ×1500, of a suture surface dried in an open air environment and a closed environment, respectively;

FIGS. 17 and 18 are graphs of a copper ion release profile with a high suture concentration and a low suture concentration, respectively; and

FIGS. 19 and 20 are graphs of a copper ion release profiles for 1C2-100 (calcium stearate) and 1C2*-100 (copper stearate), respectively.

### DETAILED DESCRIPTION OF PREFERRED EMBODIMENTS

The present invention provides improved biocompatible articles having at least one copper ion at least partially embedded therein and/or at least one copper ion at least partially coated thereon. It is appreciated that the examples described hereinbelow relate to methods for at least partially embedding copper ions within biocompatible articles, such as filaments used for sutures and manufacturing surgical meshes, as well as methods for coating filaments with copper ions and that the embedding methods and coating methods may be used alone as well as together in a wide variety of combinations.

In accordance with an embodiment of the present invention there is provided a suture including at least one filament formed of at least one polymer and a biodegradable coating including at least one copper ion, selected from copper sulfate and copper chloride, at least partially coated on the at least one filament in a manner such that the copper ion is released from the biodegradable coating over time. Polyglactin 910 sutures, either coated or uncoated, are composed of a copolymer made from 90% glycolide and 10% L-lactide.

Further in accordance with an embodiment of the present invention there is provided a suture including at least one filament formed of at least one polymer and at least one copper ion, selected from copper sulfate and copper chloride at least partially embedded in the at least one filament in a manner such that the copper ion is released from the at least one filament over time.

Additionally in accordance with a preferred embodiment of the present invention, there is provided a suture including at least one filament formed of at least PLGA and at least one copper ion at least partially embedded in the at least one filament in a manner such that the copper ion is released from the at least one filament over time.

Further in accordance with an embodiment of the present invention there is provided a master batch comprising 2-40% by weight of at least one water soluble copper compound, selected from copper sulfate and copper chloride, which may be used to produce a monofilament formed of at least one polymer and at least one copper ion, at least partially embedded in the at least one filament in a manner such that the copper ion is released from the at least one filament over time.

The following is a description of examples relating to absorbable sutures produced and operative in accordance with an embodiment of the present invention:

### General Description of Procedures

A suture with embedded antibacterial ionic particles is made as follows:

Absorbable polymers were dried in a desiccator prior to use, at 100° C. under vacuum for at least 10 hours, to reduce their water content to less than 50 ppm. The polymers included at least one of the following: aliphatic polyesters, including poly(ε-caprolactone) (PCL), polylactide (PLA), polyglycolide (PGA), polydioxanone (PDO), or copolymers thereof.

Metal particles, such as silver, zinc, copper, magnesium and cerium, are added in dry blend to the polymer and additive mixture, the size of metal particles size preferably being usually 0.2-10 micron. The particles of copper salts include: copper chloride (CuCl₂), copper sulfate (CuSO₄), and copper oxide (Cu₂O). The copper particles are pre-dried using a vacuum oven, at 120 C, under vacuum, overnight. After drying, the copper particles are ground to a particle size of 0.5-2 microns by vortex mill. The copper particles are dried prior to use, at 120° C. under vacuum for at least 10 hours.

To the resulting PLGA/copper particles compound mix, various additives are added, including plasticizers and/or stabilizers as follows:

Plasticizers

Short oligomers of aliphatic polyesters are used as plasticizers, allowing enhanced melt fluidity, and higher impact strength. Selected plasticizers alone or in combination include: Aliphatic short oligomers of Homopolymer such as Polyglycolide or Polyglycolicacid (PGA) Polylactic acid (PLA), Polycaprolactone (PCL), Polyhydroxyalkanoate (PHA), Polyhydroxybutyrate (PHB) or copolymer thereof, or copolymers of Polyethylene adipate (PEA), Polybutylene succinate (PBS). The additives were dried in a dissicator prior to use, at 40° C. under vacuum for at least 10 hours. The plasticizers are added to the polymeric composition in dry blend prior to use.

Stabilizers—Antioxidants

Additional additives are added to the polymeric composition by dry mixing prior to use. These additives include organic additives, such as Phenolic antioxidants, acting as radical scavengers which prevent thermal degradation of polymeric materials. These are combined with phosphites and thioethers to increase its effectiveness. The phosphites are efficient decomposers of hydroperoxides that are formed during the autooxidation of polymers in melt processing and the thioethers acts as secondary anti-oxidants, react with and decompose polymer peroxide to inert substances.

Stabilizers—Metal Deactivators

Metal Deactivators such as phenolic antioxidants are also added to decrease oxidative degradation that can be accelerated by copper and/or other metals present in or in contact with polymers. The addition of a metal deactivator counteracts this process and enhances the stability of the polymers.

Processing

A twin screw micro-extruder is used to melt mix the compound mixes and to draw a monofilament as described in greater detail below. In the Examples described below the processing conditions for the twin screw micro-extruder includes: temperature above the polymer melting temperature and screw speed of 50-200 RPM. The extruder is purged constantly with dry nitrogen gas. The extruder outcome is collected using a mechanical rotor with pull speed of 300-400 rpm, making uniform fiber selected thickness between 50 to 300 microns.

### Example 1

Absorbable Sutures Containing Antimicrobial Copper Sulfate Additive Embedded in Polymer.

A: Copolymer of PLGA with 90% Glycolide (G) and 10% L-Lactide (L).

The copper particles were dried prior to use, at 120° C. under vacuum for 10 hours, then sealed in an aluminum bag, under nitrogen environment. After drying, the particles were ground to a particle size of 0.5-2 microns, and the particle size was analyzed by optical microscopy. The grinding was done by vortex mill (Super Fine Ltd. Industrial Park Kidmat Galil). Copper sulfate (CuSO₄) particles of 0.5-2 micron were added in dry blend to the PLGA copolymer. A twin screw micro-extruder was used as described above, to melt mix the compound, and to draw a monofilament using 3.2 mm diameter round die head. The monofilaments were tested as described below

To the PLGA/copper particles compound various additives were added, including plasticizers and or stabilizers as follows:

Plasticizers

B: Short oligomers of poly(ε-caprolactone) with average molecular weight range of 4,000 Da (PCL 4,000), (Capa 2402, Perstorp, Sweden) were added to the polymeric composition. The oligomers were dried prior to use, at 40° C. under vacuum for at least 10 hours.

C: Short oligomers of poly(ethylene glycol) with average molecular weight of 4,000 Da (PEG 4,000). (Sigma Aldrich, Israel) was added to the PLGA/copper polymeric composition. The PEG oligomers were dried prior to use, at 40° C. under vacuum for at least 10 hours.

Stabilizers—Antioxidants

D: Selected antioxidant of Tris(nonylphenyl) phosphite (TNPP) was added to PLGA/copper composition at concentration of 0.2% wt./wt.

A structural diagram of Tris(nonylphenyl) phosphate (TNPP) is shown in FIG. 1.

Stabilizers—Metal Deactivator

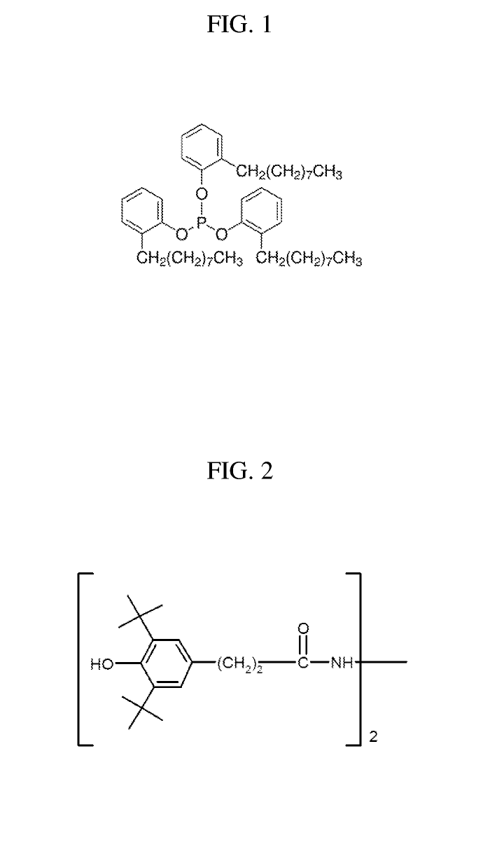

E: Metal deactivator additive of 2′, 3-bis [[3-[3, 5-di-tert-butyl-4-hydroxyphenyl] propionyl]] propionohydrazide (FIG. 2) (from Ciba, IRGANOX MD 1024, BASF Dispersions & Pigments, North America, Southfield, Mich., USA) was added to PLGA/copper composition at concentration of 0.2% wt./wt.

A structural diagram of metal deactivator (2′, 3-bis [[3-[3, 5-di-tert-butyl-4-hydroxyphenyl] propionyl]] propionohydrazide) is shown in FIG. 2.

A twin screw micro-extruder was used, as described above, to melt mix the compound, and to draw a monofilament from the compounds prepared in sub-examples A-E above. The monofilaments produced from examples 1A-E were tested as described below and the results appear thereafter.

The stabilizers selected can be used separately, or in combination, or combinations of stabilizers and plasticizers. For example: 1024 and TNPP as well as 1024 and PEG can be used in combination as described below in additional examples.

Antimicrobial Suture Analysis—General Description of Methods Used for Analysis

Mechanical Analysis

Mechanical testing of the monofilaments produced as described at A-E above was performed using Instron IX tensile tester. The tensile tester conditions include gauge length of 100 mm and the crosshead speed of 200 mm/min. according to USPHARMACOPEIA Monograph for absorbable surgical sutures appendix 881 for TENSILE STRENGTH.

Molecular Weight Analysis Using Gel Permeation Chromatograph (GPC)

The Molecular weight analysis was done using gel permeation chromatograph (GPC) of Waters 2690 Differential Separations Module equipped with differential refractometer Waters 410. The separation system is based on Styragel columns at effective molecular weight range: 100-600,000 Da. The solvent used is HPLC grade chloroform, at 1 ml/min kept at 40° C. Samples were prepared at 0.2% wt./vol. The numerical average molecular weight (Mn), weight average molecular weight (Mw) and polydispersity (PD), were calculated against polystyrene standards 3^{rd} order calibration curve.

Differential Scanning Calorimetry (DSC)

Differential scanning calorimetry (DSC) was used to Polymer analyze the transitional temperatures and crystallinity analysis. The calorimeter of Mettler TA-400, cooled using liquid nitrogen, and under inert N₂ gas environment. Analysis was done using Star-E software. Samples of 5.00-25 mg were placed in 40 μl Al-crucibles, and heating/cooling rate was 10° C./min.

Scanning Electron Microscopy (SEM) Analysis

Samples were prepared for Scanning Electron Microscopy (SEM) analysis to analyze the copper dispersion in the monofilament cross-section. The samples were sputter coated with gold and palladium (Au/Pd) using spatter coater Quorom SC716 at 12 mA for 2 minutes. The samples were then inserted to the SEM, Jeol, JSM-5410LV at 20 KV. Energy dispersive x-ray spectroscopy (EDS) of Thermo NSS7 was used to analysis the metal particles dispersion and quantity. Uncoated samples at low vacuum (LV) mode at 20 KV were used.

Suture Degradation Analysis

The composite polymeric monofilaments were immersed in phosphate buffer (PBS), and stored at 37° C., on a shaker table. Polymer degradation over time was conducted using tensile testing (Instron), molecular weight (GPC) and morphology (DSC). In addition, analysis of the released copper ions concentration was conducted.

The mechanical properties of the described combinations of compounds described in sub-examples 1A-E above, is summarized in the following Table 1.

Table 1 below summarizes the mechanical properties of selected combinations of PLGA 10/90, with copper sulfate (PLGA Cu-Sulfate), copper sulfate with plasticizers PCL (PLGA PCL Cu-Sulfate) or PEG (PLGA PEG Cu-Sulfate), copper sulfate and antioxidant TNPP (PLGA-Cu-Sulfate-TNPP) and copper sulfate and metal deactivator Irganox 1024MD (PLGA-Cu-Sulfate-1024). Note that the TNPP improved both stress and strain, as can be seen by the increased modulus.

TABLE 1

| | Stress at | Stress | | Strain | | |
|---|---|---|---|---|---|---|
| | max | at | Strain at | at | | |
| | Load | Break | max Load | Break | Modulus | Diameter |
| Compound | [MPa] | [MPa] | [%] | [%] | [MPa] | [mm] |
| PLGA | 156.1 | 133.7 | 3.0 | 2.8 | 6,211 | 0.074 |
| Cu-Sulfate | | | | | | |
| PLGA | 271.6 | 270.2 | 165.1 | 141.8 | 4,756 | 0.071 |
| PEG Cu-Sulfate | | | | | | |
| PLGA | 207.9 | 201.2 | 173.1 | 188.7 | 4,424 | 0.080 |
| PCL Cu-Sulfate | | | | | | |
| PLGA- | 590.3 | 590.4 | 221.8 | 221.4 | 11,245 | 0.055 |
| Cu-Sulfate-TNPP | | | | | | |
| PLGA- | 244.1 | 243.5 | 296.1 | 296.8 | 6,379 | 0.078 |
| Cu-Sulfate-1024 | | | | | | |

As noted above, Table 1 shows the Mechanical properties of various compounds prepared as described in Example 1A-E and analyzed as described in Example 2 Mechanical Properties. In this experiment, all copper containing compositions contained 0.5% wt/wt copper. Each analysis was conducted for at least five separate mono-filaments each of which had a diameter range of 50-90 microns.

PLGA/copper sulfate data show that TNPP improves monofilament strength and modulus. PEG improves stress and strain, while maintaining a high modulus. In preferred embodiments a combination comprising either TNPP antioxidant, or PEG plasticizer, or a combination thereof, are used. PCL improves stress, strain, and maintains a high modulus. 1024 has a positive effect on the mechanical performance including slightly higher stress, strain, and maintains a high modulus

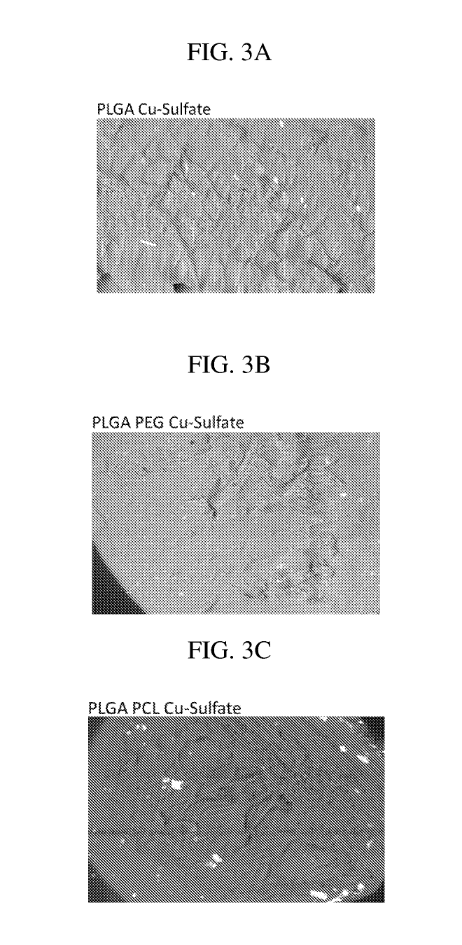

The samples were analyzed using scanning electron microscopy (SEM). The SEM micrograph of a cross-section of monofilament compounded and embedded fiber are illustrated in FIGS. 3A, 3B and 3C. The cross-sections are of PLGA 10/90, with copper sulfate (PLGA Cu-Sulfate), copper sulfate and PEG (PLGA PEG Cu-Sulfate) and copper sulfate with PCL (PLGA PCL Cu-Sulfate), respectively. It is noted that the white dots reflect the copper particles homogenously dispersed in sample PLGA PEG Cu-Sulfate, of the monofilament cross-section.

Differential Scanning Calorimetry (DSC) Analysis

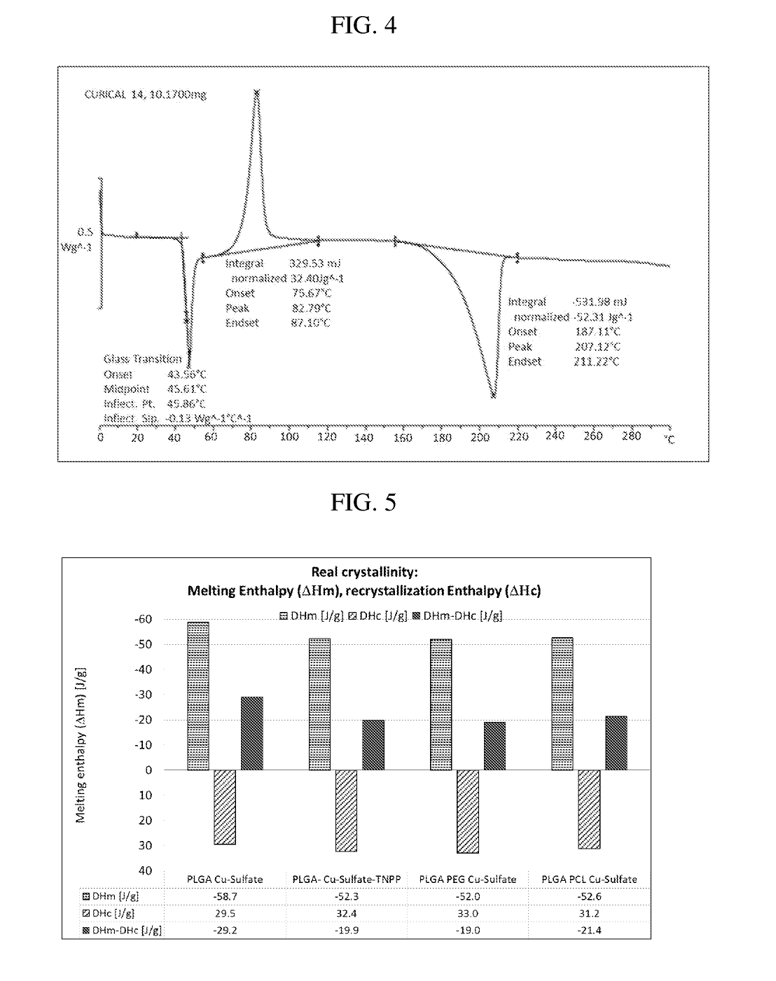

DSC was used to analyze the polymer transitional temperatures and crystallinity. FIG. 4 shows a DSC analysis of a monofilament, extruded with copper sulfate and TNPP additive. It is noted for the large Tg, the sharp exothermic peak for recrystallization, and the melting peak (Tm).

As seen in FIG. 4, the glass transitional temperature (Tg) or melting temperature (Tm), are not affected by the different additives, however, the total melting enthalpy is altered, indicating the crystallinity changes in the polymer matrix, due to the additives influence. The calculated values are summarized in FIG. 5.

FIG. 5 is a graph summarizing the enthalpy for recrystallization, melting and its difference analyzed using DSC of monofilament, extruded with copper sulfate and additives. It is noted that for the exothermic peak value for recrystallization (angled lines) at the recrystallization temperature (Tc), and the melting peak value calculated at the melting temperature (Tm) (parallel lines). The full bars are the difference between the recrystallization exotherm, and the melting endotherm, indicating for the actual crystallinity level of the polymer.

Degradation Analysis.

The polymer was immersed in deionized water or in PBS buffer at 37° C. PLGA, an aliphatic polyester, is sensitive to hydrolysis due to water molecules which initiate a nucleophilic attack on the polymer breaking it down to its monomeric units. The water molecule initially degrades the polymer's amorphous regions, and later its crystalline regions. Therefore, initially, no significant change in polymer weight, or in mechanical properties occurred, but as degradation progresses over time, the polymer collapses, and the mechanical properties were lost.

Antimicrobial Efficacy

The antimicrobial efficacy of the compositions disclosed herein and their antimicrobial activity was determined by immersing copper ion containing articles or sutures, prepared as described herein, into a saline solution containing viable bacteria, including E. coli, S. aureus, Pseudomonas aeruginosa, at a defined concentration. At given time points after immersion of the article or suture into the bacterial solution, the sample was plated on nutrient agar at various dilutions in order to calculate the amount of Colony forming units (CFUs) remaining at each time point. The calculated decrease in bacterial count in the solution provides evidence of the antibacterial activity of the copper ion releasing suture.

### Example 2

Absorbable Sutures Containing Antimicrobial Copper Oxide Additives Embedded in Polymer.

A: Copolymer of PLGA with 90% Glycolide (G) and 10% L-Lactide (L) with copper oxide.

The copper oxide particles were dried prior to use, at 120° C. under vacuum for 10 hours, then sealed in an aluminum bag, under nitrogen environment. After drying, the particles were ground to a particle size of 0.5-2 microns, the grinding was done by vortex mill and the particle size was analyzed by optical microscopy. Copper oxide (Cu₂O) particles of 0.5-1 micron were added in dry blend to the PLGA copolymer.

To the PLGA/copper oxide particles compound various additives were added, including plasticizers and or stabilizers as follows:

Plasticizers

B: Short oligomers of poly(ε-caprolactone) with average molecular weight range of 4,000 Da (PCL 4,000), (Capa 2402, Perstorp, Sweden) were added to the polymeric composition. The oligomers were dried prior to use, at 40° C. under vacuum for at least 10 hours.

C Short oligomers of poly(ethylene glycol) with average molecular weight of 4,000 Da (PEG 4,000). (Sigma Aldrich, Israel) were added to the PLGA/copper polymeric composition. The PEG oligomers were dried prior to use, at 40° C. under vacuum for at least 10 hours.

Stabilizers—Antioxidant

D: Selected antioxidant of Tris(nonylphenyl) phosphite (TNPP) (FIG. 1) was added to PLGA/copper oxide composition at concentration of 0.2% wt./wt.

Stabilizers—Metal deactivator

E: Metal deactivator additive of 2′, 3-bis [[3-[3, 5-di-tert-butyl-4-hydroxyphenyl] propionyl]] propionohydrazide (FIG. 2) (from Ciba, IRGANOX MD 1024, BASF Dispersions & Pigments, North America, Southfield, Mich., USA) was added to PLGA/copper composition at concentration of 0.2% wt./wt.

A twin screw micro-extruder was used, as described above, to melt mix the compound, and to draw a monofilament from the compounds prepared in sub examples 2A-E above. The monofilaments produced from examples 2A-E were tested as described above in Example 1 and the results appear below.

The stabilizers selected can be used separately, or in combination, or in combinations of stabilizers and plasticizers. For example, 1024 and TNPP as well as 1024 and PEG can be used in combination as described below in additional examples.

The mechanical properties of the described combinations compounds described in example 2A-E, is summarized in the Table 2.

Table 2 below summarizes the mechanical properties of selected combinations of PLGA 10/90, with copper oxide (PLGA Cu-Oxide), copper oxide with plasticizers of PCL (PLGA PCL Cu-Oxide) or PEG (PLGA PEG Cu-Oxide), copper oxide and antioxidant TNPP (PLGA-Cu-Oxide-TNPP), copper oxide and metal deactivator Irganox 1024MD (PLGA-Cu-Oxide-1024), copper oxide with antioxidant TNPP and metal deactivator of Irganox 1024MD (PLGA-Cu-Oxide-TNPP-1024). Note that the TNPP improved both stress and strain, as can be seen by the increased modulus. Also note that addition of PEG or PCL improved both stress and strain, as can be seen by the increased modulus.

TABLE 2

| | | Stress | | Strain | | |
|---|---|---|---|---|---|---|
| | Stress at | at | Strain at | at | | |
| | max Load | Break | max Load | Break | Modulus | Diameter |
| Compound | [MPa] | [MPa] | [%] | [%] | [MPa] | [mm] |
| PLGA Cu- | 212.7 | 200.4 | 161.9 | 193.7 | 6,480 | 0.076 |
| Oxide | | | | | | |
| PLGA PCL | 247.1 | 224.3 | 180.5 | 205.3 | 7,966 | 0.068 |
| Cu-Oxide | | | | | | |
| PLGA PEG | 399.9 | 293.5 | 221.0 | 218.3 | 8,574 | 0.067 |
| Cu-Oxide | | | | | | |
| PLGA-Cu- | 666.2 | 665.4 | 153.2 | 149.5 | 10,039 | 0.006 |
| Oxide-TNPP | | | | | | |
| PLGA-Cu- | 423.3 | 422.0 | 139.3 | 138.9 | 6,174 | 0.076 |
| Oxide-1024 | | | | | | |
| PLGA Cu- | 346.0 | 343.9 | 148.0 | 148.7 | 6,572 | 0.070 |
| Oxide-TNPP- | | | | | | |
| 1024 | | | | | | |

As noted above, Table 2 shows the Mechanical properties of various compounds prepared as described in Example 2A-E. In this experiment, all copper containing composition contained 0.5% wt./wt. copper. Each analysis was conducted for at least five separate mono-filaments each having a diameter range of 50-90 microns.

PLGA/copper oxide data demonstrate that TNPP improves monofilament strength and modulus. PEG improves stress and strain, while maintaining a high modulus. In preferred embodiments a combination comprising either TNPP antioxidant, or PEG plasticizer, or a combination thereof are used. PCL improves stress, strain, and maintains a high modulus. 1024 has a positive effect on the polymer mechanical performance including slightly higher stress, strain, and maintains a high modulus.

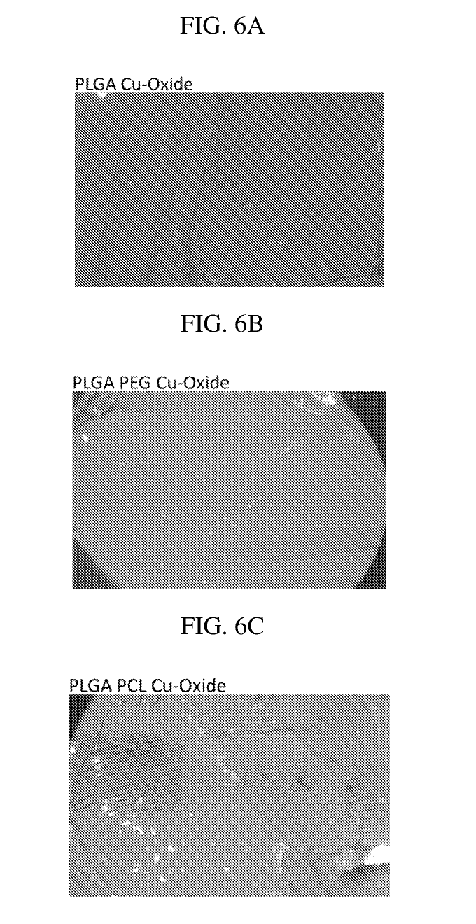

The samples were analyzed using scanning electron microscopy (SEM). The SEM micrograph of a cross-section of monofilament compounded and embedded fiber are illustrated in FIGS. 6A, 6B and 6C. The cross-sections are of PLGA 10/90, with copper oxide (PLGA Cu-Oxide), copper oxide and PEG (PLGA PEG Cu-Oxide) and copper oxide with PCL (PLGA PCL Cu-Oxide), respectively. It is noted that the white dots reflect the copper particles homogenously dispersed in sample PLGA PEG Cu-oxide, of the monofilament cross-section.

Differential Scanning Calorimetry (DSC) Analysis

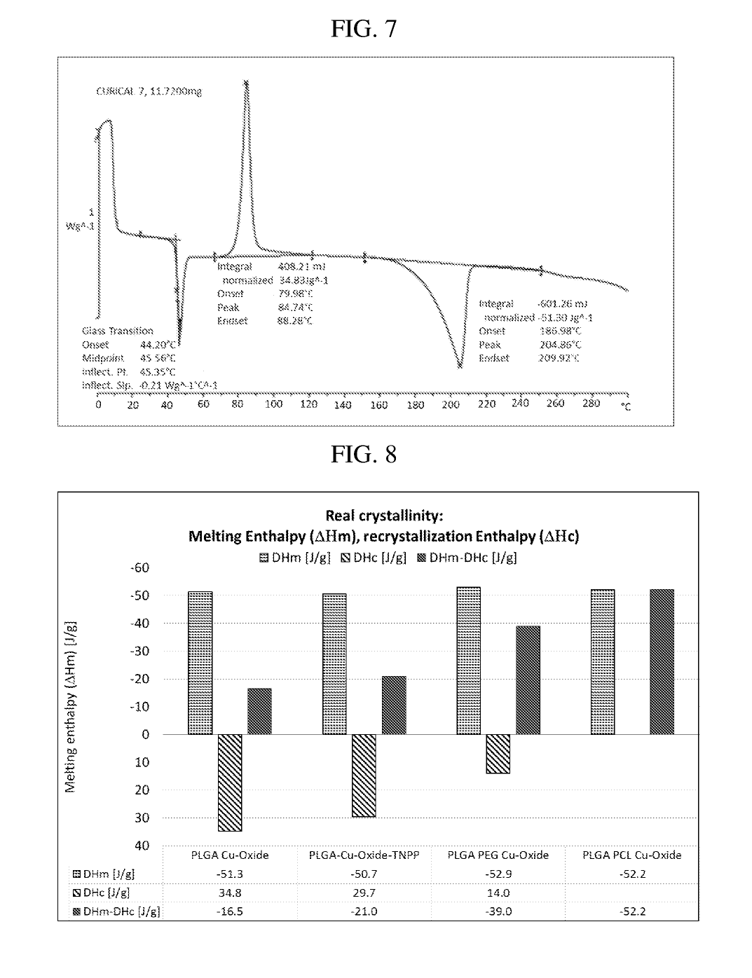

DSC was used to analyze the polymer transitional temperatures and crystallinity. FIG. 7 shows a DSC analysis of a monofilament, extruded with copper oxide. It is noted for the large Tg, the sharp exothermic peak for recrystallization (Tc), and the melting peak (Tm).

As seen in FIG. 7, the glass transitional temperature (Tg) or melting temperature (Tm), are not affected by the different additives, however, the total melting enthalpy is altered, indicating the crystallinity changes in the polymer matrix, due to the additives influence. The calculated values are summarized in FIG. 8.

FIG. 8 is a graph summarizing the enthalpy for recrystallization, melting and its difference analyzed using DSC of monofilament, extruded with copper oxide and additives. It is noted that for the exothermic peak value for recrystallization (angled lines) at the recrystallization temperature (Tc), and the melting peak value calculated at the melting temperature (Tm) (parallel lines). The full bars are the difference between the recrystallization exotherm, and the melting endotherm, indicating for the actual crystallinity level of the polymer.

Degradation Analysis.

The polymer was immersed in deionized water or in PBS buffer at 37° C. PLGA, an aliphatic polyester, is sensitive to hydrolysis due to water molecules which initiate a nucleophilic attack on the polymer breaking it down to its monomeric units. The water molecule initially degrades the polymer's amorphous regions, and later its crystalline regions. Therefore, initially, no significant change in polymer weight, or in mechanical properties occurred, but as degradation progresses over time, the polymer collapses, and the mechanical properties were lost.

### Example 3

Absorbable Sutures Containing Antimicrobial Copper Chloride Additives Embedded in Polymer.

A: Copolymer of PLGA with 90% Glycolide (G) and 10% L-Lactide (L) with copper chloride particles.

The copper particles were dried prior to use, at 120° C. under vacuum for 10 hours, then sealed in an aluminum bag, under nitrogen environment. After drying, the particles were ground to a particle size of 0.5-2 microns, the grinding was done by vortex mill and the particle size was analyzed by optical microscopy. Copper Chloride (CuCl₂) particles of 0.5-2 micron were added in dry blend to the PLGA copolymer. A twin screw micro-extruder was used, as described above, to melt mix the compound, and to draw a monofilament using 3.2 mm diameter round die head. The monofilaments were tested as described below.

To the PLGA/copper chloride particles compound various additives were added, including plasticizers and or stabilizers as follows:

Plasticizers

B: Short oligomers of poly(ε-caprolactone) with average molecular weight range of 4,000 Da (PCL 4,000), (Capa 2402, Perstorp, Sweden) were added to the polymeric composition. The oligomers were dried prior to use, at 40° C. under vacuum for at least 10 hours.

C: Short oligomers of poly(ethylene glycol) with average molecular weight of 4,000 Da (PEG 4,000). (Sigma Aldrich, Israel) was added to the PLGA/copper chloride polymeric composition. The PEG oligomers were dried prior to use, at 40° C. under vacuum for at least 10 hours.

Stabilizers—Antioxidant

D: Selected antioxidant of Tris(nonylphenyl) phosphite (TNPP) (FIG. 1) was added to PLGA/copper chloride composition at concentration of 0.2% wt./wt.

Stabilizers—Metal Deactivator

E: Metal deactivator additive of 2′,3-bis [[3-[3, 5-di-tert-butyl-4-hydroxyphenyl] propionyl]] propionohydrazide (FIG. 2) (from Ciba, IRGANOX MD 1024, BASF Dispersions & Pigments, North America, Southfield, Mich., USA) was added to PLGA/copper composition at concentration of 0.2% wt./wt.

A twin screw micro-extruder was used, as described above, to melt mix the compound, and to draw a monofilament from the compounds prepared in sub-examples 3A-E. The monofilaments produced from examples 3A-E were tested as described in Example 1 above and the results appear below.

The stabilizers selected can be used separately, or in combination, or combinations of stabilizers and plasticizers. For example, 1024 and TNPP as well as 1024 and PEG can be used in combination as described below in additional examples.

The mechanical properties of the described combinations compounds described in sub-example 3A-E above, is summarized in the following Table 3.

Table 3 below summarizes the mechanical properties of selected combinations of PLGA 10/90, with copper chloride (PLGA Cu-Chloride); copper chloride with plasticizers PCL (PLGA-PCL Cu-Chloride) or PEG (PLGA-PEG Cu-Chloride), copper chloride and antioxidant TNPP (PLGA Cu-Chloride TNPP); copper chloride and metal deactivator Irganox 1024MD (PLGA Cu-Chloride 1024); copper chloride with antioxidant TNPP and metal deactivator Irganox 1024MD (PLGA-Cu-Chloride-TNPP-1024). Note that the combination of TNPP with 1024 (PLGA-Cu-Chloride-TNPP-1024) protects both stress and strain, as can be seen by the high modulus. Also note that the PEG addition improved stress, as can be seen by the high modulus.

TABLE 3

| | | Stress | | Strain | | |
|---|---|---|---|---|---|---|
| | Stress at | at | Strain at | at | | |
| | max Load | Break | max Load | Break | Modulus | Diameter |
| Compound | [MPa] | [MPa] | [%] | [%] | [MPa] | [mm] |
| PLGA Cu-Chloride | 0.0 | 0.0 | 0.0 | 0.0 | 0 | 0.000 |
| PLGA PCL Cu-Chloride | 176.6 | 152.2 | 3.0 | 2.7 | 8,466 | 0.079 |
| PLGA PEG Cu-Chloride | 417.2 | 342.4 | 11.0 | 24.5 | 19,747 | 0.059 |
| PLGA-Cu-Chloride- | 100.6 | 95.6 | 2.9 | 3.1 | 7,245 | 0.075 |
| TNPP | | | | | | |
| PLGA-Cu-Chioride-1024 | 94.3 | 79.5 | 3.3 | 3.7 | 7,373 | 0.088 |
| PLGA-Cu-Chioride- | 188.6 | 174.6 | 216.6 | 208.9 | 7,062 | 0.069 |
| TNPP-1024 | | | | | | |

As noted above, Table 3 shows the Mechanical properties of various compounds prepared and analyzed as described in Example 3A-E. In this experiment, all copper containing composition contained 0.5% wt./wt. copper. Analysis was conducted for at least five separate mono-filaments each having a diameter range of 50-90 microns.

From the PLGA/copper chloride data, it can be seen that TNPP with 1024 maintain both stress and strain, as can be seen by the high modulus.

Also note that the addition of PEG improved stress, as can be seen by the high modulus.

In preferred embodiments a combination comprising either TNPP antioxidant, or PEG plasticizer, or a combination thereof are used. PCL has a modest positive effect on increasing stress, however reduces strain values, copper chloride results in rigid and brittle polymer. 1024 has a minor effect on the polymer's mechanical performance including a slightly higher stress and strain, as well as a high modulus.

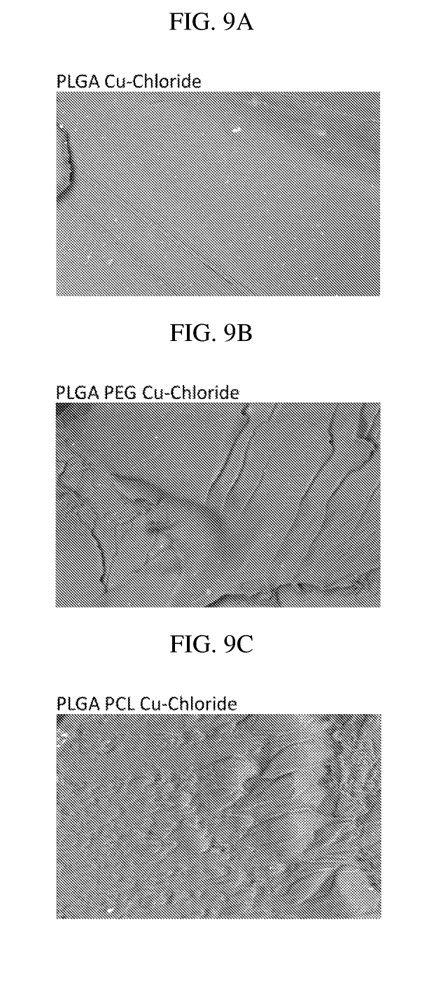

The samples were analyzed using scanning electron microscopy (SEM). The SEM micrograph of a cross-section of monofilament compounded and embedded fiber are illustrated in FIGS. 9A, 9B and 9C. The cross-sections are of PLGA 10/90, with copper chloride (PLGA Cu-chloride), copper chloride and PEG (PLGA PEG Cu-chloride), and copper chloride with PCL (PLGA PCL Cu-chloride), respectively. It is noted that the white dots reflect the copper particles homogenously dispersed in sample PLGA PEG Cu-chloride, of the monofilament cross-section.

Differential Scanning Calorimetry (DSC) Analysis

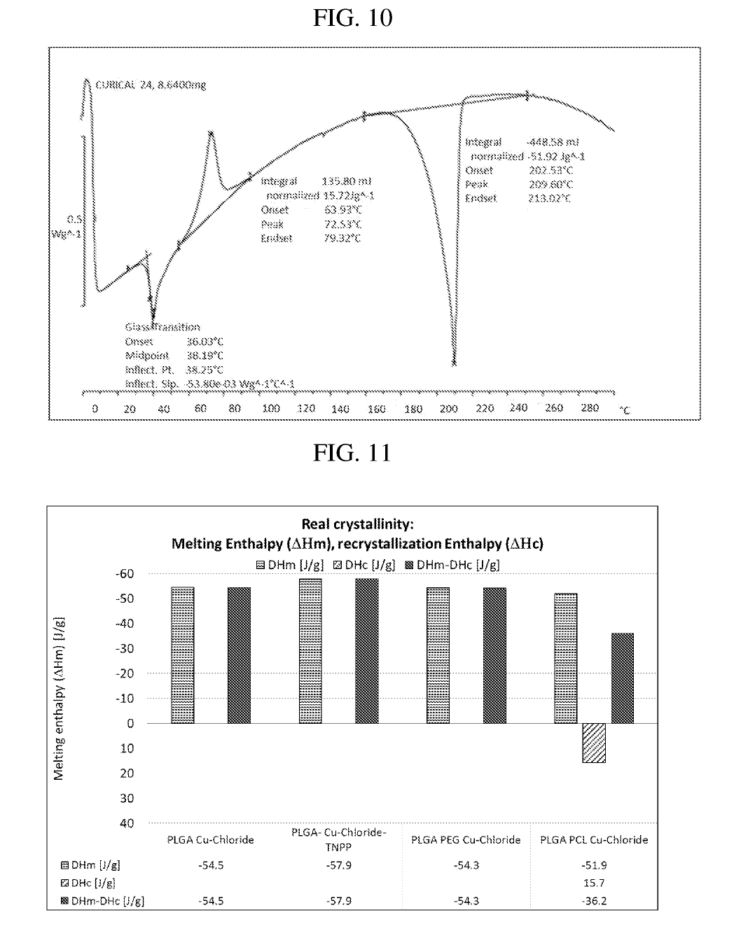

DSC was used to analyze the polymer transitional temperatures and crystallinity. FIG. 10 shows a DSC analysis of a monofilament, extruded with copper chloride with PCL additive. It is noted for the large Tg, the wide exothermic peak for recrystallization (Tc), and the melting peak (Tm).

As seen in FIG. 10, the glass transitional temperature (Tg) or melting temperature (Tm), are not affected by the different additives, however, the total melting enthalpy is altered, indicating the crystallinity changes in the polymer matrix, due to the additives influence. The calculated values are summarized in FIG. 11.

FIG. 11 is a graph summarizing the enthalpy for recrystallization, melting and its difference analyzed using DSC of monofilament, extruded with copper chloride and additives. Note that the exothermic peak value for recrystallization (angled lines) is only present in PLGA PCL Cu-chloride composition, indicating this composition's ability to recrystallize, at the recrystallization temperature (Tc), and the melting peak value calculated at the melting temperature (Tm) (parallel lines). The full bars are the difference between the recrystallization exotherm, and the melting endotherm, indicating for the actual crystallinity level of the polymer.

Preparation of a Master Batch and Processes for Preparation of a Master Batch and Filaments from a Master Batch

The following examples describe preparation of a copper containing polymer master batch and processes for preparing monofilaments and multifilament with water soluble copper compounds at least partially embedded therein from the master batch and copper containing absorbable polymer master batch and processes for preparing monofilaments and multifilament with water soluble or insoluble copper compounds at least partially embedded therein from the master batch. The examples also describe a process and master batch for preparing various products e.g. sutures and meshes comprising these monofilaments and/or multifilaments.

### BACKGROUND

Extrusion of master batch material and monofilaments was executed on a co-rotating twin screw extruder (LeistritzZSE18HPe), equipped with a Scholz gravimetric feeder system (consisting of two polymer chip feeders (type Mono) and one HETHON-FLEX HF41/51 powder feeder). The Spinneret was equipped with a 4×0.25 cubic cm per rotation spin pump or a purge plate and a 2.0 mm orifice without melt-filters depending on the particular process being performed.

General Procedures for Master Batch (MB) Production

MB Process 1A: Master Batch Prepared from Premix (One Feeder)

Water soluble Copper compounds including Copper sulfate (CS) or Copper Chloride (CC) particles are mechanically ground to a desired size (0.2-10 microns).

Polymer and copper compound are mixed and dried to form the desired percentage of a CS or CC-polymer premix (2-40% copper compound by weight).

Premix is added to the powder feeder.

Premix is extruded on a co-rotating twin screw extruder through spinneret, which may be equipped with spin pump or purge plate for lower spinneret temperatures, such as <220° C., for the Master Batch.

MB Monofilaments threads are extruded, solidified in water bath and granulated to form CS-MB or CC-MB granules.

MB Process 1B: Master Batch Prepared from Premix (One Feeder)

Insoluble Copper compounds including water insoluble Copper Oxide (CO) particles are mechanically ground to a desired size (0.2-10 microns).

Biodegradable Polymer and insoluble copper compound are mixed and dried to form the desired percentage of a CO polymer premix (2-40% copper compound by weight).

Premix is added to the powder feeder.

Premix is extruded on a co-rotating twin screw extruder through spinneret, which may be equipped with spin pump or purge plate for lower spinneret temperatures, such as <220° C., for the Master Batch.

MB Monofilaments threads are extruded, solidified in water bath and granulated to form CO-MB granules.

MB Process 2A: Master Batch Prepared from Separate Copper Compound and Pure Polymer Feeders (Dual Feeder Process):

Water soluble Copper compounds including Copper sulfate (CS) or Copper Chloride (CC) particles are ground to desired size (0.2-10 microns).

Pure Polymer is loaded into the extruder's polymer chip feeder, and ground CS or CC is loaded into the extruder via the powder feeder.

The spin pump for the polymer is started followed by the powder feeder and the mix (2-40% CS or CC) is extruded through spinneret to form MB Monofilaments threads.

Extruded MB Monofilament threads are solidified in water bath and granulated to form CS-MB or CC-MB granules.

MB Process 2B: Master Batch Prepared from Separate Copper Compound and Pure Polymer Feeders (Dual Feeder Process):

Insoluble Copper compounds including Copper Oxide (CO) particles are ground to desired size (0.2-10 microns).

Pure Biodegradable Polymer is loaded into the extruder's polymer chip feeder, and ground CO is loaded into the extruder via the powder feeder.

The spin pump for the polymer is started followed by the powder feeder and the mix (2-40% CO) is extruded through spinneret to form MB Monofilaments threads.

Extruded MB Monofilament threads are solidified in water bath and granulated to form CO-MB granules.

Monofilaments Production

MonoFilament Process 1: Monofilaments (MF) Prepared from Master Batch Granules by Premix Method (One Feeder)

Premix of CS-MB or CC-MB and pure polymer is made, for example for 5% CS-MB 77 gr and 623 gr are mixed, respectively.

Premix is added to powder feeder.

Premix is extruded through spinneret, equipped with a spin pump, to achieve target for example 0.5% CS monofilaments (MF).

Monofilaments are drawn.

Monofilaments Process 2: Monofilaments Prepared from Separate Master Batch Granules and Pure Polymer Feeders (Dual Feeder Process)

Pure Polymer is loaded into the extruder's polymer chip feeder thereafter for example 5% CS-MB or CC-MB granules are added to the extruder via the powder feeder.

The spin pump is started followed by the powder feeder and extruded through spinneret, equipped with a spin pump, to achieve target for example 0.5% CS or CC monofilaments.

Monofilaments are drawn.

Multifilament Fiber Production (e.g. Sutures) Prepared from Copper Compound-Master Batch

Multifilament Process: Multifilament Extrusion:

Take 5% CS-MB, 5% CC-MB or 5% CO-MB.

Prepare Premix with desired Copper Compound concentration, for example 0.5% copper sulfate by mixing 9-parts pure polymer with 1-part CS-MB, for example 90% pure polymer and 10% of 5% CS-MB to produce 0.5% CS-MB Premix.

Dry Premix.

Extrude Premix on a multifilament extrusion installation (single screw extruder, spin pump) and add spin finish to form spin finish filaments.

Draw the filaments with twisting to produce a drawn yarn.

Braid the drawn yarn on standard braiding machines with core and sheath yarns (core yarns are linear oriented).

Wash to remove spin finish.

Coat the braided suture (as required).

As described above, the copper compound particles or the granulated master batch material can be premixed with pure absorbable polymers such as PGLA or non-absorbable polymer to the desired copper compound concentration and then loaded into a single feeder of the extruder. Alternatively, copper compound particles or the granulated master batch material can be loaded into a first loader on the extruder and diluted by polymer which is loaded into a second loader on the extruder and diluted to the desired copper compound concentration prior to extrusion.

Furthermore, during the above described processes, MB Process 1A and MB Process 1B, a plasticizer, an antioxidant and/or a metal deactivator may be added to the premix.

Alternatively, during the above described processes MB Process 2A and MB Process 2B, an additional loader or loaders may also be used in order to add a plasticizer, an antioxidant and/or a metal deactivator to above polymeric and copper compound compositions.

Finally, the undrawn monofilaments are drawn to receive monofilaments uniform in diameter which can then optionally be braided into a multifilament.

### Example 4

Grinding of the Copper Sulfate (CuSo₄) Particles

The copper sulfate particles were dried prior to use, at 110° C. under vacuum for 10 hours, then sealed in an aluminum bag, under 99%+ nitrogen gas environment. After drying, the particles were ground to a particle size of 0.5-2.0 microns by a vortex mill (Super Fine Ltd. Industrial Park Kidmat Galil) and the particle size was analyzed by optical microscopy confirming the 0.5-2 microns particle size range.

### Example 5

### Preparation of Master Batch by Premix Process—MB Process 1

Premix Preparation—Manufacture of 5% Wt/Wt Copper Sulfate/PGLA Premix (5% CS/MB-Premix)

40 g of ground 0.5-2 microns copper sulfate (CuSO₄) particles were mixed with 760 g of 90:10-PGLA copolymer and homogenized in a glass bottle by tumbling and shaking (5% CS/MB-Premix).

Drying Procedure

The 5% CS/MB-Premix was dried at 100° C. under vacuum (<5 mbar) for at least 16 hours.

Extrusion: Extrusion of MB-Premix to Form 5% CS-MB

The dried 5% CS/MB-Premix was added to the powder feeder of the extruder in calibration mode (volumetric), and unused feeders were sealed to avoid excess humidity in the process. A purge plate (one melt channel) was used to allow for lower spinneret temperatures, e.g. 205° C. Extruder temperature zones were all 205° C.

The Master Match (MB) filaments after extrusion were solidified in a water bath with motor driven guide rollers and were taken off by a Quintett (Dienes godet system) and a tension controlled winder (Sahm 700 XE). Granulation took place on a Scheer SGS50 granulator to produce a chip length of approximately 2-3 mm.

Manufacture of the master batch 5% CS-MBvia the Premix Process had stable conditions (e.g. orifice pressure) during the extrusion time. Though Inherent viscosity was reduced from 1,442 dl/g (PGLA-Polymer) to 0,972 dl/g, this is tolerable since only ˜10% of the 5% CS-MB is mixed with ˜90% pure polymer for dilution to produce, for example, a 0.5% CS-MF.

### Example 6

### Preparation of Master Batch by Dual Feeder Process—MB Process 2

Master Batch Production with Separate Feeders for Polymer and Copper Sulfate

Drying Procedure

The PGLA and the copper sulfate particles were dried at 100° C. under vacuum (<5 mbar) for at least 16 hours.

Extrusion: Co-Extrusion of Polymer and Copper Sulfate to Form 5% CS-MB

The dried pure 90:10 PGLA polymer was added to the polymer chip feeder of the extruder in automatic mode. The copper sulfate was added to the powder feeder. Spin pump rotation was calculated to ˜20 rpm to reach a copper sulfate concentration of ˜5% in the MB for a throughput of 95 g/h of the powder feeder.

The temperature of the extruder zones were between 220° C.-235° C. and the temperature of the spinneret with the spin pump was set about 220-235° C. The trial was started with pure polymer (without feeding the copper compound) to bring the polymer feeder into a steady state concerning the pre-pressure in front of the spin pump. Subsequently, the powder feeder with the addition of copper sulfate was started.

The master batch monofilaments after extrusion are solidified in a water bath with motor driven guide rollers and are taken off by a Quintett (Dienes godet system) and a tension controlled winder (Sahm 700 XE). Granulation is performed on a Scheer SGS50 granulator to produce a chip length of approximately 2-3 mm.

Monofilament Production

### Example 7

MF Process 1: Monofilaments Prepared from Master Batch Granules by Premix Method (One Feeder)

Monofilament Preparation: Premix Preparation of 0.5% CS-MF-Premix from the 5% CS-MB

77 g of the above described 5% wt/wt MB (5% CS-MB) granules were added to 623 g of pure copolymer 90:10 PGLA (a hypothetical 9 fold dilution of the copper concentration) (0.5% CS-MF-Premix) and homogenized in a glass bottle by tumbling and shaking and dried at 100° C. under vacuum (<5 mbar) for at least 16 hours to reduce humidity in the extrusion process as detailed above.

Pure PGLA (w/o Copper Sulfate) was used to manufacture pure PGLA monofilaments (PGLA-MF) under comparable conditions as a reference for comparative analysis.

Extrusion of Monofilaments

A single screw extruder (two-zone Ankele, VE1-18-20-6) was used for the monofilament extrusion of 0.5% wt/wt copper loaded PGLA monofilaments (0.5% CS-MF) from 0.5% CS-MF-Premix and the pure PGLA (PGLA-MF) as a reference.

The extrusion of PGLA-MF (pure PGLA monofilaments) was performed with a 220° C. spinneret temperature using a spin pump. The extrusion of 0.5% wt/wt copper loaded PGLA monofilaments (0.5% CS-MF) was done at two different spinneret temperatures; 220° C. and 235° C., 0.5% CS-MF-220° C. and 0.5% CS-MF-235° C.), respectively. The extruder zone temperatures were 205° C./210° C. for PGLA-MF and 0.5% CS-MF-220° C. and 205° C./220° C. for 0.5% CS-MF-235° C. Orifice was 1.25 mm.

Extrusion and drawing were executed as separate processes.

Monofilaments Drawing Process

The extruded monofilaments drawing process for PGLA-MF, 0.5% CS-MF-220° C. and 0.5% CS-MF-235° C., was performed in continuous furnaces (Erge, length=1.5 m) of the Dienes godet system to produce PGLA-MFD, 0.5% CS-MFD-220° C. and 0.5% CS-MFD-235° C.

Diameter of the drawn monofilaments was measured by the double axis laser measurement system ODAC15XY with the processor unit USYS 20-0100-A (Zumbach).

Non-drawn monofilaments with a diameter of ˜0.53 mm±0.02 mm were drawn to produce monofilaments having a diameter of ˜0.2 mm.

The different parameters for the drawn monofilaments are detailed in Table 4, where: Q1, Q2 and Q3 are the velocities of Quintetts (godets) 1 to 3;

TABLE 4

| | Q1 | | Q2 | | Q3 | | | | | Std-dev | |
|---|---|---|---|---|---|---|---|---|---|---|---|
| Sample name | [m/min] | T1 [° C.] | [m/min] | T2 [° C.] | [m/min] | DR1 | DR2 | DR | d [mm] | [mm] | Ov [mm] |
| PGLA-MFD | 4.1 | 65 | 26 | 80 | 27 | 6.34 | 1.04 | 6.59 | 0.205 | 0.006 | 0.001 |
| 0.5% CS-MFD-220° C.-V1 | 4.1 | 65 | 26 | 80 | 27 | 6.34 | 1.04 | 6.59 | 0.204 | 0.011 | 0.002 |
| 0.5% CS-MFD-220° C.-V2 | 3.8 | 65 | 26 | 80 | 27 | 6.84 | 1.04 | 7.11 | 0.199 | 0.007 | 0.001 |
| 0.5% CS-MFD-235° C.-V1 | 4.1 | 65 | 26 | 80 | 27 | 6.34 | 1.04 | 6.59 | 0.204 | 0.020 | 0.004 |
| 0.5% CS-MFD-235° C.-V2 | 3.6 | 65 | 26 | 80 | 27 | 7.22 | 1.04 | 7.50 | 0.193 | 0.006 | 0.001 |

T1 and T2 are furnace temperatures;
DR is the draw ratio;
Ovality (Ov) is the averaged difference between x- and y-axis measured by the double axis laser measurement system ODAC15XY with a USYS 20-0100-A (Zumbach) processor unit.

The 0.5% CS-MFD-220° C.-V1 and 0.5% CS-MFD235° C.-V2 were drawn under the same conditions as PGLA-MFD, but showed a higher diameter standard deviation. Increasing draw ratio (V2) reduced the standard deviation similar to PGLA-MFD.

In order to control and minimize the standard deviation of the drawn monofilament diameter the draw can be increased as can be seen in table 4.

### Example 8

Analysis of MB and MF Materials—Composition and Residual Monomer by NMR Spectroscopy

5-10 mg of the drawn monofilaments (PGLA-MFD, 0.5% CS-MFD-220° C.) and 0.5% CS-MFD-235° C.) were dissolved in 0.35 ml Trifluoro Acetic Acid (TFE), and then 0.65 ml of chloroform (CDCl3) was added. The solution was transferred to a 5 mm NMR tube and samples were measured with a Bruker Fourier 300 spectrometer. Polymer composition (mol-%) was calculated from the lactide methyl group peak at 1.65 ppm and from the glycolide peak at 4.90 ppm.

Residual lactide monomer is calculated from the peak at 1.71 ppm. Results are in % mol in relation to the total polymer composition and indicate that this process has not caused any significant polymer degradation. The results are summarized in Table 5.

TABLE 5

| | L-lactide | Glycolide | RM Lactide |
|---|---|---|---|
| Sample name | [% mol] | [% mol] | [% mol] |
| PGLA-MFD | 9.7 | 90.3 | 0.7 |
| 0.5% CS-MFD-220° C. | 9.7 | 90.3 | 0.8 |
| 0.5% CS-MFD-235° C. | 9.7 | 90.3 | 0.9 |

RM = residual monomer

Inherent Viscosity

200 mg from each of 1) the pure copolymer 90:10 PGLA, 2) 5% CS-MB and drawn monofilaments (PGLA-MFD, 0.5% CS-MFD-220° C. and 0.5% CS-MFD—235° C.) were dissolved in 25 ml hexafluoro-isopropanol (HFIP, c=0.8 g/dl).

After filtration, the solution was transferred into an Ubbelohde capillary Oa. Measurements were made at 30° C. with a Schott AVS370 system.

Inherent viscosity: iV=ln (t/t0)/c. Correction of flow times (t and t0) was done by Hagenbach.

The inherent viscosity for the monofilaments are summarized in Table 6.

TABLE 6

| | i.v |
|---|---|
| Sample name | [dl/g] |
| PGLA polymer | 1.442 |
| 5% CS-MB | 0.972 |
| PGLA-MFD | 1.346 |
| 0.5% CS-MFD-220° C. | 1.260 |
| 0.5% CS-MFD-235° C. | 1.247 |

The measured inherent viscosity of pure 90:10 PGLA is 1.442 dl/g. The 5% CS-MBhad a reduction of the iV to 0.972 dl/g (reduction of 32.6%). The 9-10 times diluted 0.5% CS-MFD-220° C. and 0.5% CS-MFD-235° C. drawn monofilaments had a slightly lower iV values than the PGLA-MFD.

Extrusion of monofilaments from pure 90:10 PGLA had a Iv of 1.346 dl/g, while monofilaments of PGLA loaded with 0.5% copper sulfate as described above have iV of 1.260 dl/g and 1.247 dl/g for 0.5% CS-MFD-220° C. and 0.5% CS-MFD-235° C., respectively.

Adding copper sulfate to PGLA results in reduction of 6.39% in the monofilament iV for 0.5% CS-MFD-220° C. and reduction of 7.35% in the monofilament iV for 0.5% CS-MFD-235° C. compare to monofilaments from pure 90:10 PGLA.

The small reduction in the inherent viscosity (˜7%) for the copper loaded monofilaments is insignificant with respect to degradation of the 0.5% CS-MFD.

Copper Concentration by Inductively Coupled Plasma Optical Emission Spectrometry (ICP-OES)

ICP-OES method was used to analyze the actual amount of copper in the polymeric monofilaments after the extrusion process. 3 types of copper loaded polymeric monofilaments were analyzed (5% CS-MB, 0.5% CS-MFD-220° C. and 0.5% CS-MFD-235° C.).

The above samples were digested in 5 ml of HNO3 65% and 1 mL of HCl 37%. Digestion was carried out in quartz vessels using a “Discover” sample digestion system at high temperature and high pressure (CEM, USA). Vessels were cooled down and the volume was made up to 20 mL with deionized water. All samples were dissolved completely. Element concentration was measured in the clear solutions using an axial ICP-OES model ‘ARCOS’ from Spectro GMBH, Germany. Measurements were calibrated with standards for ICP from Merck. Element concentrations that exceeded the linear dynamic range were diluted and reanalyzed. Dilution was made using calibrated pipettes. The continuing calibration verification standard was measured to check the instrument stability.

Table 7 shows the calculated amount of copper (Cu) element in the above samples and was used to calculate the weight percent of the copper sulfate (CuSO₄) in the monofilaments. Results are summarized in Table 7.

TABLE 7

| | Cu | CuSO₄ |
|---|---|---|
| Sample name | [mg/kg] | [% wt] |
| 5% CS-MB | 22064 | 5.54 |
| 0.5% CS-MFD-220° C. | 1571 | 0.39 |
| 0.5% CS-MFD-235° C. | 1800 | 0.45 |

The ICP results indicate that the amount of copper sulfate in the 5% CS-MB material and the drawn monofilaments 0.5% CS-MFD-235° C. is not significantly lower (64-73% of theoretical concentration) than the loading dose prior to the extrusion process.

Mechanical Analysis: Stress-Strain Test

Mechanical testing of the monofilaments produced as described at above were measured linear and with a single knot on a Zwick UPM 1435 ZMART.PRO universal test machine with Test-Expert II evaluation software. The tensile tester conditions include gauge length of 80 mm and the crosshead speed of 200 mm/min.

Table 8 summarizes the different mechanical tests for the monofilaments after drawing.

TABLE 8

| | Average | | | | | | |
|---|---|---|---|---|---|---|---|
| | diameter | LTS | LTS | L-Elong. | KPTS | KPTS | K-Elong. |
| Sample name | [mm] | [N] | [N/mm²] | [%] | [N] | [N/mm²] | [%] |
| PGLA-MFD | 0.205 | 17.9 | 542 | 18.3 | 11.3 | 342 | 13.4 |
| 0.5% CS-MFD-220° C.-V1 | 0.204 | 19.6 | 598 | 34.0 | 10.4 | 320 | 21.5 |
| 0.5% CS-MFD-220° C.-V2 | 0.199 | 16.7 | 540 | 15.5 | 9.8 | 314 | 9.3 |
| 0.5% CS-MFD-235° C.-V1 | 0.204 | 18.6 | 569 | 47.2 | 10.8 | 329 | 37.3 |
| 0.5% CS-MFD-235° C.-V2 | 0.193 | 19.5 | 665 | 30.7 | 7.0 | 238 | 12.9 |

LTS = Linear Tensile Strength;
KPTS = Knot Pull Tensile Strength;
L-Elong./K-Elong. = Elongation at break for linear and knot pull test.

Samples 0.5% CS-MFD-220° C.-V1 and 0.5% CS-MFD-235° C.-V1 that were drawn at a lower draw ratios than 0.5% CS-MFD-220° C.-V2 and 0.5% CS-MFD-235° C.-V2, showed higher linear elongation at break values

The LTS values of the copper loaded monofilaments and pure PGLA monofilaments are similar, indicating that no or little agglomeration of copper sulfate within the polymeric matrix which would otherwise weaken the monofilament.

The KPTS values of the copper loaded monofilaments are similar to the pure PGLA monofilaments. 0.5% CS-MFD (at 235° C.)-V2 showed insignificantly lower KPTS values.

### Example 9

Multifilament Fiber Production (e.g. Sutures) Prepared from CS-Master Batch

Multifilament Process: Multifilament Extrusion:

Take 5% CS-MB, 5% CC-MB or 5% CO-MB.

Prepare Premix with desired Copper Compound concentration for example 0.5% copper sulfate by mixing 9-parts pure polymer with 1-part CS-MB (for example 90% pure polymer and 10% 5% CS-MB to produce 0.5% CS-MB Premix).

Dry Premix.

Extrude Premix on a multifilament extrusion installation (single screw extruder, spin pump) and add spin finish to form spin finish filaments.

Draw the filaments with twisting to produce a drawn yarn.

Braid the drawn yarn on standard braiding machines with core and sheath yarns (core yarns are linear oriented).

Wash to remove spin finish.

Coat the braided suture (as required).

Process parameters: The above process may incorporate the following steps and apparatus:

Possible extruder is an Extruder Fa. Barmag, Typ E 1 Nr. 10/6248 with winder Baby ASW, Typ MSW-50S-72Z

Melt filters with high mesh number and low pore size (preferably pore size ˜40 μm somewhat larger than the drawn single filament diameter which is normally only in the range of 10-30 μm however smaller pore diameter sizes may also be used)

Single orifice capillary diameter is ˜250 μm. One orifice contains a high number of capillaries (e.g. 30)

No water bath for solidification, a vertical spin chamber with a significant height is used

Spin finish is used to prevent electrostatic charging of the braided yarn and to hold the single filaments of the yarn together (spin finish is removed after the braiding process (before coating). The spin finish is added below the orifice.

Spin draft and winder speed (e.g. 1000 m/min)

Drawing is done with twisting on specialized drawing machines such as an Edmund Erdmann, DMT 24/200-6

Spools for braiding process: Hacoba, Typ FSA

Braiding: Steeger, Typ ERT 5 with 8, 12 or 16 spool braiding tables (depending on desired braid construction)

Antimicrobial Non-Absorbable Sutures

General Description of Procedures

Suture with embedded antibacterial ionic particles are made as follows:

Polymers are dried in a desiccator prior to use, at 100° C. under vacuum for at least 10 hours, to reduce water content to less than 200 ppm. The polymers used are one of the following non-absorbable polymers including: nylon, polyester, polyvinylidene fluoride (PVDF) and polypropylene (PP).

Metal particles, such as silver, zinc, copper, magnesium and cerium, are added in dry blend to the polymer and additive mixture, the metal particles size is usually 0.2-10 micron, copper salts particles include; copper chloride (CuCl₂) and copper sulfate (CuSO₄). The copper particles are pre-dried using vacuum oven, at 120° C., under vacuum, overnight. After drying, the particles are ground to a particle size of 0.5-2 microns by vortex mill. The copper particles are dried again prior to use, at 120° C. under vacuum for at least 10 hours.

To the polymer/copper particles compound various additives are added, including plasticizers and or stabilizers as following:

Plasticizers

A plasticizer is selected from the group consisting of stearic acid and calcium stearate, the plasticizer being present in the mixture in an amount from about 0.001 to about 5 percent by weight. The plasticizers are added to the polymeric composition in dry blend prior to use.

Other Additives

Other additives may also be present on and/or within the fiber substrate, including antistatic agents, nucleating agents, antioxidants, UV stabilizers, fillers, softeners, lubricants, curing accelerators, and the like. All of such additional materials are well known to those skilled in the art and are commercially available.

Processing

A twin screw extruder is used to melt mix the compound, and to draw a monofilament. The processing conditions for the twin screw extruder included: a temperature above the polymer melting temperature and a screw speed of 50-400 RPM. The extruder is degas sed constantly to remove volatiles.

The extruder outcome is collected using a mechanical rotor with pull speed of 300-400 rpm, making uniform fiber selected thickness between 20 to 150 microns.

The monofilament fiber is composed of combinations thereof.

### Example 10

Antimicrobial Non-Absorbable Polyamide (PA6,6) Sutures Containing Antimicrobial Copper Sulfate (CuSO₄) Additives Embedded in Polymer.

A: In a specific example, polymer of polyamide (PA6,6) is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper sulfate (CuSO₄) particles of 0.5-2 micron are added in dry blend to the Nylon 6,6 polymer. A twin screw extruder is used to melt mix the compound, and to draw a monofilament using round die head.

To the Nylon/copper particles compound different additives can be added, including plasticizers and or stabilizers as following:

Plasticizers

B: Stearic acid is added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: Selected antioxidant of solid organophosphate is added to the composition as stabilizer at Nylon/copper composition at concentration of 0.2% wt./wt.

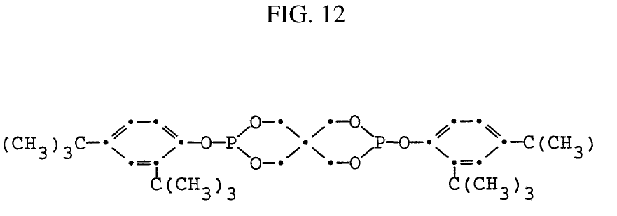

A structural diagram of solid organophosphate ULTRANOX 626 phosphite Antioxidant of Bis(2,4-di-tert-butylphenyl) pentaerythritoldiphosphite is shown in FIG. 12.

The processing conditions for the twin screw micro-extruder for sub-examples 10A-D include: temperature profiles of 240° C., 250° C. and 260° C. along the extruder heating zones, and a screw speed of 50-400 RPM. The extruder is degassed constantly to remove volatiles.

The extruder outcome is collected using a mechanical rotor with a pull speed of 300-400 rpm, making uniform fiber selected thickness between 20 to 150 microns.

### Example 11

Antimicrobial Non-Absorbable Polyamide (PA6,6) Sutures Containing Antimicrobial Copper Chloride (CuCl₂) Additives Embedded in Polymer.

A: In a specific example, polymer of polyamide (PA6,6) is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper chloride (CuCl₂) particles of 0.5-2 micron are added in dry blend to the Nylon 6,6 polymer. A twin screw extruder is used to melt mix the compound, and to draw a monofilament using round die head.

To the Nylon/copper particles compound different additives can be added, including plasticizers and or stabilizers as following:

Plasticizers

B: Stearic acid is added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: Selected antioxidant of solid organophosphate is added to the composition as stabilizer at Nylon/copper composition at concentration of 0.2% wt./wt.

The processing conditions for the twin screw micro-extruder for examples 11A-D include: temperature profiles of 240° C., 250° C. and 260° C. along the extruder heating zones, and a screw speed of 50-400 RPM. The extruder is degassed constantly to remove volatiles

The extruder outcome is collected using a mechanical rotor with a pull speed of 300-400 rpm, making uniform fiber selected thickness between 20 to 150 microns.

### Example 12

Antimicrobial Non-Absorbable Polyethylene Terephthalate (PET) Sutures Containing Antimicrobial Copper Sulfate (CuSO₄) Additives Embedded in Polymer.

A: In a specific example, polymer of polyethylene terephthalate (PET) polyester is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper sulfate (CuSO₄) particles of 0.5-2 micron are added in dry blend to the PET polymer. A twin screw extruder is used to melt mix the compound, and to draw a monofilament using round die head.

To the PET/copper particles compound various additives are added, including plasticizers and or stabilizers as follows:

Plasticizers

B: Stearic acid is added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: selected antioxidant of solid organophosphate is added to the composition as stabilizer at PET/copper composition at concentration of 0.2% wt./wt.

The processing conditions for the twin screw micro-extruder for examples 12A-D include: temperature profiles of 265° C., 275° C. and 280° C. along the extruder heating zones, and a screw speed of 50-400 RPM. The extruder is degassed constantly to remove volatiles.

The extruder outcome is collected using a mechanical rotor with a pull speed of 300-400 rpm, making uniform fiber selected thickness between 20 to 150 microns.

### Example 13

### Antimicrobial Non-Absorbable Polyethylene Terephthalate (PET) Sutures Containing Antimicrobial Copper Chloride (CuCl₂) Additives Embedded in Polymer

A: in a specific example, polymer of polyethylene terephthalate (PET) polyester is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper chloride (CuCl₂) particles of 0.5-2 micron are added in dry blend to the PET polymer. A twin screw extruder is used to melt mix the compound, and to draw a monofilament using round die head.

To the PET/copper particles compound various additives are added, including plasticizers and or stabilizers as follows:

Plasticizers

B: Stearic acid i added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: selected antioxidant of solid organophosphate is added to the composition as stabilizer at PET/copper composition at concentration of 0.2% wt./wt.

The processing conditions for the twin screw micro-extruder for examples 13A-D include: temperature profiles of 265° C., 275° C. and 280° C. along the extruder heating zones, and a screw speed of 50-400 RPM. The extruder is degassed constantly to remove volatiles.

The extruder outcome is collected using a mechanical rotor with a pull speed of 300-400 rpm, making uniform fiber selected thickness between 20 to 150 microns.

### Example 14

Antimicrobial Non-Absorbable Polyvinylidene Fluoride (PVDF) Sutures Containing Antimicrobial Copper Sulfate (CuSO₄) Additives Embedded in Polymer.

A: In a specific example, polymer of polyvinylidene fluoride (PVDF) is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper sulfate (CuSO₄) particles of 0.5-2 micron are added in dry blend to the PVDF polymer. A twin screw extruder is used to melt mix the compound, and to draw a monofilament using round die head.

To the PVDF/copper particles compound various additives are added, including plasticizers and or stabilizers as follows:

Plasticizers

B: Stearic acid is added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: selected antioxidant of solid organophosphate is added to the composition as stabilizer at PVDF/copper composition at concentration of 0.2% wt./wt.

The processing conditions for the twin screw micro-extruder for examples 14A-D include: temperature profiles of 220° C., 230° C. and 240° C. along the extruder heating zones, and a screw speed of 50-400 RPM. The extruder is degassed constantly to remove volatiles.

The extruder outcome is collected using a mechanical rotor with a pull speed of 300-400 rpm, making uniform fiber selected thickness between 20 to 150 microns.

### Example 15

Antimicrobial Non-Absorbable Polyvinylidene Fluoride (PVDF) Sutures Containing Antimicrobial Copper Chloride (CuCl₂) Additives Embedded in Polymer.

A: In a specific example, polymer of polyvinylidene fluoride (PVDF) is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper chloride (CuCl₂) particles of 0.5-2 micron are added in dry blend to the PVDF polymer. A twin screw extruder is used to melt mix the compound, and to draw a monofilament using round die head.

To the PVDF/copper particles compound various additives are added, including plasticizers and or stabilizers as following:

Plasticizers

B: Stearic acid is added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: selected antioxidant of solid organophosphate is added to the composition as stabilizer at PVDF/copper composition at concentration of 0.2% wt./wt.

The processing conditions for the twin screw micro-extruder for examples 15A-D include: temperature profiles of 220° C., 230° C. and 240° C. along the extruder heating zones, and a screw speed of 50-400 RPM. The extruder is degassed constantly to remove volatiles.

The extruder outcome is collected using a mechanical rotor with a pull speed of 300-400 rpm, making uniform fiber selected thickness between 20 to 150 microns.

### Example 16

### Antimicrobial Non-Absorbable Polypropylene (PP) Sutures Containing Antimicrobial Additives Copper Sulfate (CuSO₄) Embedded in Polymer

A: In a specific example, polymer of polypropylene (PP) is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size was analyzed by optical microscopy. Copper sulfate (CuSO₄) particles of 0.5-2 micron are added in dry blend to the PP polymer. A twin screw extruder is used to melt mix the compound, and to draw a monofilament using round die head.

To the PP/copper particles compound various additives are added, including plasticizers and or stabilizers as follows:

Plasticizers

B: Stearic acid is added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: selected antioxidant of solid organophosphate is added to the composition as stabilizer at PP/copper composition at concentration of 0.2% wt./wt.

The processing conditions for the twin screw micro-extruder for examples 16A-D include: temperature profiles of 210° C., 220° C. and 230° C. along the extruder heating zones, and a screw speed of 50-400 RPM. The extruder is degassed constantly to remove volatiles.

The extruder outcome is collected using a mechanical rotor with a pull speed of 300-400 rpm, making uniform fiber selected thickness between 20 to 150 microns.

### Example 17

Antimicrobial Non-Absorbable Polypropylene (PP) Sutures Containing Antimicrobial Additives Copper Chloride (CuCl₂) Embedded in Polymer.

A: In a specific example, polymer of polypropylene (PP) is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper chloride (CuCl₂) particles of 0.5-2 micron are added in dry blend to the PP polymer. A twin screw extruder is used to melt mix the compound, and to draw a monofilament using round die head.

To the PP/copper particles compound various additives are added, including plasticizers and or stabilizers as following:

Plasticizers

B: Stearic acid is added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: selected antioxidant of solid organophosphate is added to the composition as stabilizer at PP/copper composition at concentration of 0.2% wt./wt.

The processing conditions for the twin screw micro-extruder for examples 17A-D included: temperature profiles of 210° C., 220° C. and 230° C. along the extruder heating zones, and a screw speed of 50-400 RPM. The extruder is degassed constantly to remove volatiles.

The extruder outcome is collected using a mechanical rotor with a pull speed of 300-400 rpm, making uniform fiber selected thickness between 20 to 150 microns.

Surgical Degradable Mesh

General Description of Procedures

These embodiments of the invention relate to synthetic, bioabsorbable polymer materials and implants, like fibers, sutures, meshes and other tissue management, wound closure or tissue engineering devices. These embodiments of the invention also relate to methods of preventing and treating infections by using synthetic, bioabsorbable aliphatic polyesters, including poly(ε-caprolactone) (PCL), polylactide (PLA), polyglycolide (PGA), polydioxanone (PDO), or copolymers thereof.

Antimicrobial Additives

Metal particles such as silver, zinc, copper, magnesium and cerium, are added in dry blend to the polymer and additive mixture, the metal particles size is usually 0.2-10 micron. The particles of copper salts includes; copper chloride (CuCl₂), copper sulfate (CuSO₄), and copper oxide (Cu₂O). The copper particles are pre-dried using vacuum oven, at 120° C., under vacuum, overnight. After drying, the particles are grounded to a particle size of 0.5-2 microns by vortex mill. The copper particles are dried prior to use, at 120° C. under vacuum for at least 10 hours.

Surgical devices prepared from extruded materials include mesh prostheses conventionally used to repair hernias. Such mesh fabric prostheses are also used in other surgical procedures, including the repair of anatomical defects of the abdominal wall, diaphragm, and body walls, correction of defects in the genitourinary system, and repair of traumatically damaged organs, such as the spleen, liver or kidney or in inducing the formation of fibrous tissue small joint in fingers of rheumatoid patients (U.S. Pat. No. 6,113,640) or as scaffolds for tissue engineering (Gaissmaer et al. 2002, Länsman et al. 2002).

In a preferred embodiment of the present invention, the devices have a surface that is antimicrobial, yet does not interfere with wound healing to the extent that affects clinical outcome adversely. The multifunctional devices of an embodiment of the present invention can be made in any appropriate form to contain a polymer matrix and antibiotic(s), employing polymer technological processing methods. Typical forms are mono- and/or multifilamentous sutures and their derivatives such as meshes and scaffolds.

Mesh Manufacturing

The device, e.g. sutures or mesh, can be manufactured from bioabsorbable fibers using any of the known methods from mechanical textile and plastics technology. The thickness of the fibers can vary from about 1 micrometer to about 200 micrometers. In a preferred embodiment of the invention, the fiber thickness is between ca. 5 micrometers and ca. 150 micrometers.

Structures suitable for making the multifunctional device, wherein the device is a mesh, can be, for example, a cloth, a narrow fabric, a knit, a weave, a braid, or a web. In any of these cases, the structure should be porous with a pore size from ca 30 micrometers to ca 1000 micrometers, preferably between ca. 50 micrometers to ca. 400 micrometers. The mesh can be manufactured using one type of fiber, for example PGA or PLA or their copolymeric fibers. It is also possible to make the mesh using two or more different types of fibers depending on the particular application and desired physical characteristics of the implant. The mesh can be manufactured using both bioabsorbable and non-bioabsorbable fibers.

The multifunctional device (mesh) can be manufactured by employing known and conventional warp knitting apparatus and techniques, such as the tricot and Raschel knitting machines and procedures described in “Warp Knitting Production” by Dr. S. Raz, MelliandTextilberichte GmbH, Rohrbacher Str. 76, D-6900 Heidelberg, Germany (1987).

The fibers are melt-spun with a twin-screw extruder, where the polymer melt temperatures range from 200° C. to 240° C. and are pressed through round die holes having diameter of e.g. about 0.4 mm. After cooling, filaments are oriented freely in a two-step process at elevated temperature, first at 60° C. to 140° C. to a draw ratio of e.g. 4 to 8. The final filament diameter can be 50 micrometers. The filaments are knitted by using a weft-knitting machine, with the fabric having loop size ca. 1 mm.

Following knitting, the mesh is cleaned or scoured, and thereafter annealed to stabilize the fabric. For the latter operation, the mesh can be secured to a tenter frame which maintains the mesh at a predetermined width, the frame then being passed through an elongated heating zone. Following heat setting, the mesh is cut to size, packaged and sterilized.

The mesh can be cut to any desired configuration, e.g. a square or rectangular shape of appropriate dimensions. An ultrasonic slitter, various types of which are commercially available, may be employed to cut the mesh. Unlike the result one may obtain when cutting with a blade, i.e. frayed yarn ends, or when the yarn ends are heat-sealed, i.e. bead-like formations, the ultrasonic slitter cuts the mesh to the desired size.

A multifunctional mesh device can have two types of filaments, e.g. bioabsorbable and non-bioabsorbable. The pharmacological agent is included in the bioabsorbable filament. For example, a non-bioabsorbable polypropylene monofilament exhibits good pliability. Depending on the material used to form the mesh, a mesh preferably has adequate flexibility. In addition, depending on the yarn used to form the mesh, a mesh formed preferably has a sufficient burst strength.

### Example 18

Absorbable Mesh Containing Antimicrobial Additives Embedded in Polymer.

A: In a specific example, copolymer of PLGA with 90% Glycolide (G) and 10% L-Lactide (L).

The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper sulfate (CuSO₄) particles of 0.5-2.0 micron are added in dry blend to the PLGA copolymer. A twin screw extruder is used to melt mix the compound, and to draw a monofilament using 3.2 mm diameter round die head.

To the PLGA/copper particles compound various additives are added, including plasticizers and or stabilizers as follows:

Plasticizers

B: Short oligomers of poly(ε-caprolactone) with average molecular weight range of 4,000 Da (PCL 4,000), (Capa 2402, Perstorp, Sweden) are added to the polymeric composition. The oligomers are dried prior to use, at 40° C. under vacuum for at least 10 hours.

C: Short oligomers of poly(ethylene glycol) with average molecular weight of 4,000 Da (PEG 4,000). (Sigma Aldrich, Israel) are added to the PLGA/copper polymeric composition. The PEG oligomers are dried prior to use, at 40° C. under vacuum for at least 10 hours.

Stabilizers—Antioxidant

D: Selected antioxidant of Tris(nonylphenyl) phosphite (TNPP) is added to PLGA/copper composition at concentration of 0.2% wt./wt.

Stabilizers—Metal deactivator

E: Metal deactivator additive of 2′, 3-bis [[3-[3, 5-di-tert-butyl-4-hydroxyphenyl] propionyl]] propionohydrazide is added to PLGA/copper composition at concentration of 0.2% wt./wt.

The processing conditions for the twin screw extruder for examples 18A-E include: temperature profiles of 200° C., 205° C. and 210° C. along the extruder heating zones, and a screw speed of 50-200 RPM. The extruder is purged constantly with dry nitrogen gas.

The extruder outcome is collected using a mechanical rotor with a pull speed of 300-400 rpm, making uniform fiber selected thickness between 20 to 150 microns.

### Example 19

Antimicrobial Non-Absorbable Mesh Containing Antimicrobial Additives Embedded in Polymer.

A: In a specific example, polymer of polyamide (PA6,6) or Nylon 6,6, is dried using a desiccator at 60° C. for 8 hours, until dew point of −40° C. is reached. The copper particles are dried prior to use, at 120° C. under vacuum for 10 hours, then are sealed in an aluminum bag, under nitrogen environment. After drying, the particles are ground to a particle size of 0.5-2 microns, the grinding is done by vortex mill and the particle size is analyzed by optical microscopy. Copper sulfate (CuSO₄) particles of 0.5-2 micron are added in dry blend to the Nylon 6,6 polymer. A twin screw extruder is used to melt mix the compound, and to draw a monofilament using a round die head.

To the Nylon/copper particles compound various additives are added, including plasticizers and or stabilizers as following:

Plasticizers

B: Stearic acid is added to the polymeric composition. The plasticizer is dried prior to use, at 35° C. under vacuum for at least 10 hours.

C: Calcium stearate is added to the polymeric composition. The plasticizer is dried prior to use, at 35° C. under vacuum for at least 10 hours.

Stabilizers—Antioxidant

D: Selected antioxidant of solid organophosphate, is added to the composition as stabilizer at Nylon/copper composition at concentration of 0.2% wt./wt.

The processing conditions for the twin screw micro-extruder for examples 19A-D include: temperature profiles of 240° C., 250° C. and 260° C. along the extruder heating zones, and a screw speed of 50-400 RPM. The extruder is degassed constantly to remove volatiles.

The extruder outcome is collected using a mechanical rotor with a pull speed of 300-400 rpm, making uniform fiber selected thickness between 20 to 150 microns.

Antimicrobial Surgical Glue

General Description

A wide range of treatments are applicable, including wound treatment and other medical procedures. For example, surgical glue can be used as a replacement for, or in addition to, sutures or staples to join together two surfaces. The material can also be used to coat, protect, or otherwise cover surface, superficial, internal, or topical wounds including, but not limited to, minor cuts, scrapes, irritations, compromised skin, superficial lacerations, abrasions, burns, sores, and stomatitis. The material composition can also be used on undamaged tissues for local delivery or release of antimicrobial entities to a patient through healthy tissue.

A medical glue composition typically contains cyanoacrylate derivatives, including methyl cyanoacrylate, ethyl cyanoacrylate, butyl cyanoacrylate, octyl cyanoacrylate or any modified cyanoacrylate. Antimicrobial copper particles of size of 0.5-2.0 microns of copper oxide, copper sulfate and copper chloride are added. In addition, stabilizers of organic acid (formic, acetic, propionic or citric) and sulfur dioxide are added to the formulation.

### Example 20

A medical glue having the composition provided herein comprises copper particles at weight concentration of 0.05-5.5% wt./wt., N-butyl-2-cyanoacrylate 93.5-99.8% wt./wt. and at least one stabilizer organic acid (formic, acetic, propionic or citric) 0.1-0.4% wt./wt. and sulfur dioxide 0.1-0.6% wt./wt.

N-butyl-2-cyanoacrylate, cooled to 4° C., is mixed with the required quantity of organic acid under an inert environment of nitrogen or argon purge. The required quantity of copper particles, including copper oxide, copper sulfate and copper chloride or combinations thereof, are placed into a container containing sulfur dioxide, the mixture is then stirred until homogenous dispersion of the copper particles are formed.

The copper particles act as anti-inflammatory and antimicrobial component in the composition.

N-butyl-2-cyanoacrylate acts as the adhesive binder in the composition.

Antimicrobial Bone Cement

General Description

Bone cement is used for orthopedic hard tissue repair. The cement is based on acrylic components, such that the cured cement contains poly(methacrylic acid esters).

A typical bone cement mixture, pre-polymer, contains an acrylic copolymer powder, for example a poly(methyl-methacrylate)/styrene copolymer, an acrylic monomer, for example methyl-methacrylate, in which the weight ratio of polymer to monomer is 2:1.

Bone cements, whether used for fixing implants in hard tissue or as fillers for repair purposes, are generally required to remain in place for many years and therefore need to be non-degradable and inert in body fluids. The onset and propagation of bacterial infections in such regions cause loosening of the implant or the repair, swelling, pain and general discomfort and may ultimately require more radical treatment such as amputation of an affected limb.

Anti-bacterial or anti-microbial bone cements are used by incorporating therein a bactericide. Copper particles release copper ions with antimicrobial activity. The antimicrobial activity via release of copper ions can be achieved using different copper ion releasing particles, including copper oxide, copper sulfate and copper chloride.

The antimicrobial composition described herein, for incorporation in bone cement mixtures, gives a sustained antimicrobial effect even in aggressive environments and/or when incorporated in certain resins or polymers which tend to mask or destroy the effect.

### Example 21

Copper sulfate particles are pre-dried using a vacuum oven, at 120° C., under vacuum overnight. After drying, the particles are ground to a particle size of 0.5-2.0 microns by vortex mill. The copper particles are dried prior to use, at 120° C. under vacuum for at least 10 hours. The particles are loaded to the pre-polymer composition at 0.05-5.5% wt./wt. then, the cement mixture of poly(methyl-methacrylate)/styrene copolymer, and the acrylic monomer of methyl-methacrylate, are mixed thoroughly, until homogeneous dispersion is formed.

Bone cement compositions according to the invention exhibit mechanical and curing properties which are within the essential limits laid down in ASTM F 451 part 46.

The antimicrobial bone cements comprising an acrylic polymer and a copper particles filler, are also radiopaque, and can be used for imaging.

The following is a description of further examples relating to absorbable sutures produced and operative in accordance with an embodiment of the present invention:

Preparation of Coating Solutions

Copper Chloride

Overview of Process Using Solution 1A:

65:35 P(D,L)LGA polymer [(LACTEL Absorbable Polymers, USA) Lot no 1143-21-01] was dissolved in ethyl acetate [(Bio Lab, Israel) Cat no 05400521] and then calcium stearate [(Sigma Aldrich, Israel) CAS no 1592-23-0] OR copper stearate [(MP-Bio, USA) Cat no 211952] was dissolved in the polymer ethyl acetate solution producing a uniform solution in which small particles were visible. Copper chloride [(CuCl₂) (MP-Bio, USA) Cat no 205185] was dissolved in a separate acetone [(Bio Lab, Israel) Cat no 010305] solution to a concentration of 2.5% wt/wt. The two solutions were then mixed together at a ratio of 1:1.

Preparation of a 2%, 5% and 10% wt/wt solution wherein the % represents the total amount of solids in solution including 65:35 P(D,L)LGA copolymer and calcium stearate or copper stearate as the case may be (excluding the copper compound):

Overview of Process Using Solution 1B:

65:35 P(D,L)LGA polymer [(LACTEL Absorbable Polymers, USA) Lot no 1143-21-01] was dissolved in acetone[(Bio Lab, Israel) Cat no 010305] and then calcium stearate [(Sigma Aldrich, Israel) CAS no 1592-23-0] OR copper stearate [(MP-Bio, USA) Cat no 211952] was dissolved in the polymer acetone solution producing a uniform solution in which small particles were visible. Copper chloride [(CuCl₂) (MP-Bio, USA) Cat no 205185] was added to the above solution and mixed on a stirrer for additional 1 hr.

Preparation of a 2%, 5% and 10% wt/wt solution whereas the % represents the total amount of solids in solution including 65:35 P(D,L)LGA copolymer and calcium stearate or copper stearate as the case may be (excluding the copper compound):

Overview of Process Using Solution 1C:

65:35 P(D,L)LGA polymer [(LACTEL Absorbable Polymers, USA) Lot no 1143-21-01] and calcium stearate [(Sigma Aldrich, Israel) CAS no 1592-23-0] OR copper stearate [(MP-Bio, USA) Cat no 211952] and Copper chloride [(CuCl₂) (MP-Bio, USA) Cat no 205185] were mixed and dissolved together in methyl ethyl ketone MEK (2-Butanone) EMPLURA No. 1.06014.6025 (Merck kGaA) [Synonyms for MEK are 2-butanone, ethyl methyl ketone, and methyl acetone] using a magnetic stirrer, at room temperature for overnight, producing a uniform suspension in which small particles were visible.

Preparation of a 2%, 5% and 10% wt/wt solutions (solution 1C1, 1C2 and 1C3, respectively) whereas the % represents the total amount of solids in solution including 65:35 P(D,L)LGA copolymer and calcium stearate or copper stearate as the case may be (excluding the copper compound).

2% Wt/Wt Coating Solutions

Control Coating Solution—2% Wt/Wt:

0.4 g of the copolymer 65:35 P(D,L)LGA was added to 39.2 g of ethyl acetate or acetone in a glass container. Ethyl acetate and acetone are solvents that evaporated rapidly even at room temperature (RT). Therefore, the glass container was covered with aluminum foil to control solvent evaporation. Evaporation rate of chemicals are reported in comparison to Butyl acetate whose evaporation rate is standardized as 1.0. Chemicals with evaporation rate of 3 times higher than Butyl acetate, such as acetone and ethyl acetate, are classified as having rapid evaporation rates. The copolymer solution was mixed on stirrer at RT until compounds were fully dissolved and solution was clear.

0.4 g of calcium stearate or copper stearate was added to solution and mixed for an additional 1 hr.

1A1 Copper Chloride Solution 1A—2% wt/wt:

Solution 1:

0.4 g of the copolymer 65:35 P(D,L)LGA was added to 19.2 g of ethyl acetate in a glass container. The glass container was covered with aluminum foil to prevent rapid solvent evaporation. Solution was mixed on stirrer at RT until compounds were fully dissolved and solution was clear.

0.4 g of calcium stearate or copper stearate was added to solution and mixed for 1 hr. 20 gr total.

Solution 2:

0.5 g of copper chloride (CuCl₂) with 19.5 g acetone was stirred separately for 30 min at RT until clear solution. 20 gr total

Solution 1 and 2 were mixed together for an additional 15 min. under RT, covered with aluminum foil.

1B1 Copper Chloride Solution 1B—2% Wt/Wt:

0.4 g of the copolymer 65:35 P(D,L)LGA was added to 38.7 g of acetone in a glass container. The glass container was covered with aluminum foil to prevent rapid solvent evaporation. Solution was mixed on stirrer at RT until compounds were fully dissolved and solution was clear.

0.4 g of calcium stearate or copper stearate was added to solution and mixed for 1 hr.

0.5 g of copper chloride was added to the solution and mixed for additional 1 hr.

5% Wt/Wt Coating Solutions

Control Coating Solution—5% Wt/Wt:

1 g of the copolymer 65:35 P(D,L)LGA was added to 38 g of ethyl acetate or acetone in a glass container. Ethyl acetate and acetone are solvents that evaporated rapidly even at RT. Therefore, the glass container was covered with aluminum foil to control solvent evaporation. Evaporation rate of chemicals are reported in comparison to Butyl acetate whose evaporation rate is standardized as 1.0. Chemicals with evaporation rate of 3 times higher than Butyl acetate, such as acetone and ethyl acetate, are classified as having rapid evaporation rates. The copolymer solution was mixed on stirrer at RT until compounds were fully dissolved and solution was clear.

1 g of calcium stearate or copper stearate was added to solution and mixed for an additional 1 hr.

1A2 Copper Chloride Solution 1A—5% Wt/Wt:

Solution 1:

1 g of the copolymer 65:35 P(D,L)LGA was added to 18 g of ethyl acetate in a glass container. The glass container was covered with aluminum foil to prevent rapid solvent evaporation. Solution was mixed on stirrer at RT until compounds were fully dissolved and solution was clear.

1 g of calcium stearate or copper stearate was added to solution and mixed for 1 hr. 20 gr total

Solution 2:

0.5 g of copper chloride (CuCl₂) with 19.5 g acetone was stirred separately for 30 min at RT until clear solution. 20 gr total

Solution 1 and 2 were mixed together for an additional 15 min. under RT, covered with aluminum foil.

1B2 Copper Chloride Solution 1B—5% Wt/Wt:

1 g of the copolymer 65:35 P(D,L)LGA was added to 37.5 g of acetone in a glass container. The glass container was covered with aluminum foil to prevent rapid solvent evaporation. Solution was mixed on stirrer at RT until compounds were fully dissolved and solution was clear.

1 g of calcium stearate or copper stearate was added to solution and mixed for 1 hr.

0.5 g of copper chloride was added to the solution and mixed for additional 1 hr.

10% Wt/Wt Coating Solutions

Control Coating Solution—10% Wt/Wt:

2 g of the copolymer 65:35 P(D,L)LGA was added to 36 g of ethyl acetate or acetone in a glass container. Ethyl acetate and acetone are solvents that evaporated rapidly even at RT. Therefore, the glass container was covered with aluminum foil to control solvent evaporation. Evaporation rate of chemicals are reported in comparison to Butyl acetate whose evaporation rate is standardized as 1.0. Chemicals with evaporation rate of 3 times higher than Butyl acetate, such as acetone and ethyl acetate, are classified as having rapid evaporation rates. The copolymer solution was mixed on stirrer at RT until compounds were fully dissolved and solution was clear.

2 g of calcium stearate or copper stearate was added to solution and mixed for an additional 1 hr.

1A3 Copper Chloride Solution 1A—10% Wt/Wt:

Solution 1:

2 g of the copolymer 65:35 P(D,L)LGA was added to 16 g of ethyl acetate in a glass container. The glass container was covered with aluminum foil to prevent rapid solvent evaporation. Solution was mixed on stirrer at RT until compounds were fully dissolved and solution was clear.

2 g of calcium stearate or copper stearate was added to solution and mixed for 1 hr. 20 gr total

Solution 2:

0.5 g of copper chloride (CuCl₂) with 19.5 g acetone was stirred separately for 30 min at RT until clear solution. 20 gr total

Solution 1 and 2 were mixed together for an additional 15 min. under RT, covered with aluminum foil.

1B3 Copper Chloride Solution 1B—10% Wt/Wt:

2 g of the copolymer 65:35 P(D,L)LGA was added to 35.5 g of acetone in a glass container. The glass container was covered with aluminum foil to prevent rapid solvent evaporation. Solution was mixed on stirrer at RT until compounds were fully dissolved and solution was clear.

2 g of calcium stearate or copper stearate was added to solution and mixed for 1 hr.

0.5 g of copper chloride was added to the solution and mixed for additional 1 hr.

Copper Sulfate

Overview of Process Using Solution 2A:

65:35 P(D,L)LGA polymer [(LACTEL Absorbable Polymers, USA) Lot no 1143-21-01] was dissolved in ethyl acetate [(Bio Lab, Israel) Cat no 05400521] and then calcium stearate [(Sigma Aldrich, Israel) CAS no 1592-23-0] OR copper stearate [(MP-Bio, USA) Cat no 211952] was dissolved in the polymer ethyl acetate solution producing a uniform solution in which small particles were visible. Copper sulfate [(CuSO₄) (Sigma Aldrich, Israel) CAS no 7758-98-7] was added to the above solution and mixed on a stirrer for additional 1.5 hrs.

Preparation of a 2%, 5% and 10% wt/wt solution whereas the % represents the total amount of solids in solution including 65:35 P(D,L)LGA copolymer and calcium stearate or copper stearate as the case may be (excluding the copper compound):

2% Wt/Wt Coating Solutions

Control Coating Solution—2% Wt/Wt:

0.4 g of the copolymer 65:35 P(D,L)LGA was added to 39.2 g of ethyl acetate or acetone in a glass container. Ethyl acetate and acetone are solvents that evaporated rapidly even at RT. Therefore, the glass container was covered with aluminum foil to control solvent evaporation. Evaporation rate of chemicals are reported in comparison to Butyl acetate whose evaporation rate is standardized as 1.0. Chemicals with evaporation rate of 3 times higher than Butyl acetate, such as acetone and ethyl acetate, are classified as having rapid evaporation rates. The copolymer solution was mixed on stirrer at RT until compounds were fully dissolved and solution was clear.

0.4 g of calcium stearate or copper stearate was added to solution and mixed for an additional 1 hr.

2A1 Copper Sulfate Solution 2A—2% Wt/Wt:

0.4 g of the copolymer 65:35 P(D,L)LGA was added to 38.7 g of ethyl acetate or acetone in a glass container. The glass container was covered with aluminum foil to prevent rapid solvent evaporation. Solution was mixed on stirrer at RT until compounds were fully dissolved and solution was clear.

0.4 g of calcium stearate or copper stearate was added to solution and mixed for 1 hr.

0.5 g of copper sulfate was added to the solution and mixed for additional 1.5 hrs.

5% Wt/Wt Coating Solutions

Control Coating Solution—5% Wt/Wt:

1 g of the copolymer 65:35 P(D,L)LGA was added to 38 g of ethyl acetate or acetone in a glass container. Ethyl acetate and acetone are solvents that evaporated rapidly even at RT. Therefore, the glass container was covered with aluminum foil to control solvent evaporation. Evaporation rate of chemicals are reported in comparison to Butyl acetate whose evaporation rate is standardized as 1.0. Chemicals with evaporation rate of 3 times higher than Butyl acetate, such as acetone and ethyl acetate, are classified as having rapid evaporation rates. The copolymer solution was mixed on stirrer at RT until compounds were fully dissolved and solution was clear.

1 g of calcium stearate or copper stearate was added to solution and mixed for an additional 1 hr.

2A2 Copper Sulfate Solution 2A—5% wt/wt:

1 g of the copolymer 65:35 P(D,L)LGA was added to 37.5 g of ethyl acetate or acetone in a glass container. The glass container was covered with aluminum foil to prevent rapid solvent evaporation. Solution was mixed on stirrer at RT until compounds were fully dissolved and solution was clear.

1 g of calcium stearate or copper stearate was added to solution and mixed for 1 hr.

0.5 g of copper sulfate was added to the solution and mixed for additional 1.5 hrs.

10% wt/wt coating solutions

Control coating solution—10% wt/wt:

2 g of the copolymer 65:35 P(D,L)LGA was added to 36 g of ethyl acetate or acetone in a glass container. Ethyl acetate and acetone are solvents that evaporated rapidly even at RT. Therefore, the glass container was covered with aluminum foil to control solvent evaporation. Evaporation rate of chemicals are reported in comparison to Butyl acetate whose evaporation rate is standardized as 1.0. Chemicals with evaporation rate of 3 times higher than Butyl acetate, such as acetone and ethyl acetate, are classified as having rapid evaporation rates. The copolymer solution was mixed on stirrer at RT until compounds were fully dissolved and solution was clear.

2 g of calcium stearate or copper stearate was added to solution and mixed for an additional 1 hr.

2A3 Copper Sulfate Solution 2A—10% Wt/Wt:

2 g of the copolymer 65:35 P(D,L)LGA was added to 35.5 g of ethyl acetate or acetone in a glass container. The glass container was covered with aluminum foil to prevent rapid solvent evaporation. Solution was mixed on stirrer at RT until compounds were fully dissolved and solution was clear.

2 g of calcium stearate or copper stearate was added to solution and mixed for 1 hr.

0.5 g of copper sulfate was added to the solution and mixed for additional 1.5 hrs.

Table 9 summarizes the components of the different coating solutions prepared as described above for copper chloride and copper sulfate in different coating solution percentages of 2%, 5% and 10% wt/wt solution, where the % represents the total amount of solids in solution including 65:35 P(D,L)LGA copolymer and calcium stearate or copper stearate as the case may be (excluding the copper compound).

TABLE 9

| | 65:35 | Calcium | Copper | Copper | Copper | Ethyl | |
|---|---|---|---|---|---|---|---|
| % Coating | PDLLGA | stearate | stearate | chloride | sulfate | acetate | Acetone |
| solution | [g] | [g] | [g] | (CuCl2) [g] | (CuSO4) [g] | [g] | [g] |
| 2%- | 0.4 | 0.4 | — | — | — | 39.2 | — |
| Control | 0.4 | 0.4 | — | — | — | — | 39.2 |
| | 0.4 | — | 0.4 | — | — | 39.2 | — |
| | 0.4 | — | 0.4 | — | — | — | 39.2 |
| 2%-Cupric | 0.4 | 0.4 | — | 0.5 | — | 19.2 | 19.5 |
| chloride | 0.4 | — | 0.4 | 0.5 | — | 19.2 | 19.5 |
| | 0.4 | 0.4 | — | 0.5 | — | — | 38.7 |
| | 0.4 | — | 0.4 | 0.5 | — | — | 38.7 |
| 2%-Cupric | 0.4 | 0.4 | — | — | 0.5 | 38.7 | — |
| sulfate | 0.4 | 0.4 | — | — | 0.5 | — | 38.7 |
| | 0.4 | — | 0.4 | — | 0.5 | 38.7 | — |
| | 0.4 | — | 0.4 | — | 0.5 | — | 38.7 |
| 5%- | 1 | 1 | — | — | — | 38 | — |
| Control | 1 | 1 | — | — | — | — | 38 |
| | 1 | — | 1 | — | — | 38 | — |
| | 1 | — | 1 | — | — | — | 38 |
| 5%-Cupric | 1 | 1 | — | 0.5 | — | 18 | 19.5 |
| chloride | 1 | — | 1 | 0.5 | — | 18 | 19.5 |
| | 1 | 1 | — | 0.5 | — | — | 37.5 |
| | 1 | — | 1 | 0.5 | — | — | 37.5 |
| 5%-Cupric | 1 | 1 | — | — | 0.5 | 37.5 | — |
| sulfate | 1 | 1 | — | — | 0.5 | — | 37.5 |
| | 1 | — | 1 | — | 0.5 | 37.5 | — |
| | 1 | — | 1 | — | 0.5 | — | 37.5 |
| 10%- | 2 | 2 | — | — | — | 36 | — |
| Control | 2 | 2 | — | — | — | — | 36 |
| | 2 | — | 2 | — | — | 36 | — |
| | 2 | — | 2 | — | — | — | 36 |
| 10%- | 2 | 2 | — | 0.5 | — | 16 | 19.5 |
| Cupric | 2 | — | 2 | 0.5 | — | 16 | 19.5 |
| chloride | 2 | 2 | — | 0.5 | — | — | 35.5 |
| | 2 | — | 2 | 0.5 | — | — | 35.5 |
| 10%- | 2 | 2 | — | — | 0.5 | 35.5 | — |
| Cupric | 2 | 2 | — | — | 0.5 | — | 35.5 |
| sulfate | 2 | — | 2 | — | 0.5 | 35.5 | — |
| | 2 | — | 2 | — | 0.5 | — | 35.5 |

### Example 22: Coating a Suture with Coating Solution

Uncoated polyglactin 910 sutures, composed of copolymer made from 90% glycolide and 10% L-lactide, were cut into 10 cm segments and weighed (W1).

Coating Procedure:

Uncoated polyglactin 910 suture segments were dipped in the Coating Solutions prepared as described above for 5 seconds. Subsequently, using a Delicate Task Wiper (Kimtech by Kimberly-Clark) excess material was wiped from the suture as it was removed from the coating solution.

Solvent Evaporation:

wiped sutures were hung for 72 hrs in a closed glass environmental chamber to control solvent evaporation rates.

Annealing:

Evaporated sutures were placed in a pre-heated oven at 110° C. for 10 minutes. After 10 minutes the oven was turned off and sutures were left to cool in the oven until the oven reached RT.

Sutures were then removed from oven and weighed (W2). Coating weight was calculated by the following equation: % coating=(W2−W1)/W1*100%

Process Parameters and Effects on Coating Characteristics

The following parameters were evaluated:

Copper Chloride vs. Copper Sulfate;

The effect of different coating solution weight percentage (2%, 5% and 10% wt/wt);

The effect of dipping time of the suture in the coating solutions (5, 10 and 15 sec);

The effect of drying the coated suture in an open air environment versus a closed environment. Providing a closed environment allows greater control of the solvent evaporation rate in order to reduce the surface porosity to ensure smoother surface.

The effects of the different parameters on the suture coating is detailed hereinbelow in the SEM analysis section.

Analysis of the Sutures:

Mechanical Analysis:

Mechanical testing was performed using Instron IX tensile tester. The tensile tester conditions includes gauge length of 100 mm and the crosshead speed of 200 mm/min. according to USP Monograph for absorbable surgical sutures appendix 881 for TENSILE STRENGTH.

The mechanical properties of the coated sutures described in example 1A2 and its control solution and the control solution of example 1A3 compared to sutures before coating (uncoated sutures) and to the common commercial products; coated Vicryl (same coating as in our control samples) and Vicryl Plus (coated with Triclosan) are summarized in Table 10 below.

All suture samples have the same diameter size of USP 3-0.

Table 10 shows the mechanical properties of different suture types: the commercial sutures (1 and 2), an uncoated suture (3), suture coated with coating solution without copper at 5% and 10% wt/wt (4 and 5, respectively) and sutures coated with copper chloride solution of 5% (6).

TABLE 10

| | | Stress at | Stress at | Strain at | Strain at |
|---|---|---|---|---|---|
| | | max Load | break | max Load | break |
| | Suture Type | [Mpa] | [Mpa] | [%] | [%] |
| 1 | Coated Vicryl | 46.43 | 46.36 | 26.16 | 26.24 |
| 2 | Vicryl Plus | 45.41 | 45.31 | 24.91 | 25.04 |
| 3 | Uncoated | 36.08 | 36.08 | 26.02 | 25.98 |
| 4 | CONTROL_5% wt/wt | 37.46 | 37.46 | 34.43 | 34.43 |
| 5 | CONTROL_10% wt/wt | 38.57 | 38.56 | 35.03 | 35.07 |
| 6 | Copper chloride_5% | 37.24 | 37.24 | 33.19 | 33.19 |
| | wt/wt | | | | |

The results indicate that all the tested sutures meet the USP monograph for absorbable sutures requirements: minimum tensile strength of USP 3-0 sutures not less than 17.4N.

The process of coating the sutures with copper does not change the suture mechanical properties.

*) Differences in values between the commercial sutures, i.e. Vicryl and Vicryl Plus, to the coated sutures are due to different suture manufacturers.

SEM (Scanning Electron Microscopy) Analysis:

Samples were prepared for Scanning Electron Microscopy (SEM) analysis to analyze the suture coating by observing the suture surface. The samples were sputter coated with gold and palladium (Au/Pd) using spatter coater Quorom SC716 at 12 mA for 2 minutes. Then the samples were inserted to the SEM, Jeol, JSM-5410LV at 20 KV.

Energy dispersive x-ray spectroscopy (EDS) of Thermo NSS7 was used to analyze the presence of copper in the coating, uncoated samples at low vacuum (LV) mode at 20 KV were used.

Different Coating Solution Weight Percentage:

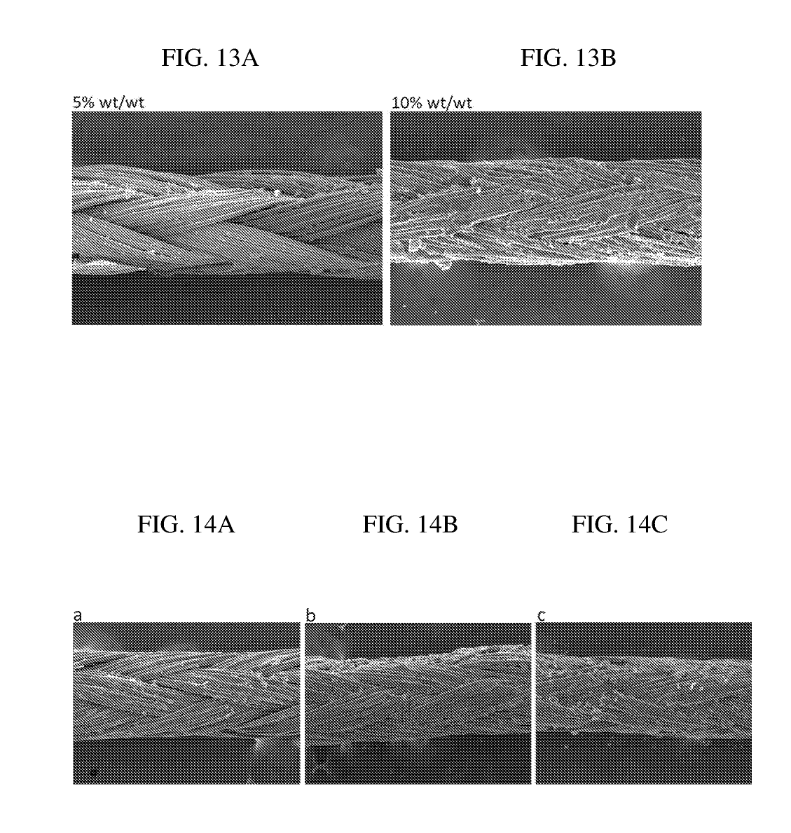

As seen in FIGS. 13A and 13B, higher weight percentage of coating solution results in a denser coating of the suture multifilaments. FIGS. 13A and 13B show a suture surface with a coating solution of 5% wt/wt (as described in examples 1A2, 1B2 and 2A2) and coating solution of 10% wt/wt (as described in examples 1A3, 1B3 and 2A3), respectively.

Suture Dipping Time in the Coating Solution:

As seen in FIGS. 14A, 14B and 14C, for the same coating solution as described above at 1A1, 1A2 and 1A3 or at 2A1, 2A2 and 2A3(2%, 5% or 10% wt/wt), the dipping time of the suture in the coating solution also influences the suture coverage. The longer the contact time of the suture in the coating solution, the denser the resulting coating is as can be seen in FIGS. 14A-14C. The coating in FIG. 14A, with a dipping time of 5 seconds, is more uniform, with a minimal aggregation of particles on the surface, than the coatings of FIGS. 14B and 14C, where a longer dipping time of the suture in the coating solution results in a thicker coating with aggregation of particles on the suture surface. As seen from a comparison of FIG. 14B, with a dipping time of 10 seconds, and FIG. 14C, with a dipping time of 15 seconds, the longer the dipping time the greater the particle aggregation on the surface. As seen, FIG. 14B shows fewer surface particles than FIG. 14C. The lower aggregation of particles and therefore increased smoothness is advantageous since it allows for easier passage of the suture in the tissue.

Coating the Sutures with Copper Chloride Vs. Copper Sulfate:

The copper chloride is fully dissolved in acetone (until its saturation point is reached), while the copper sulfate particles are dispersed in acetone and ethyl acetate. Therefore, a coating solution with copper sulfate has larger particles than a coating solution with copper chloride. These particles tend to precipitate after a few minutes.

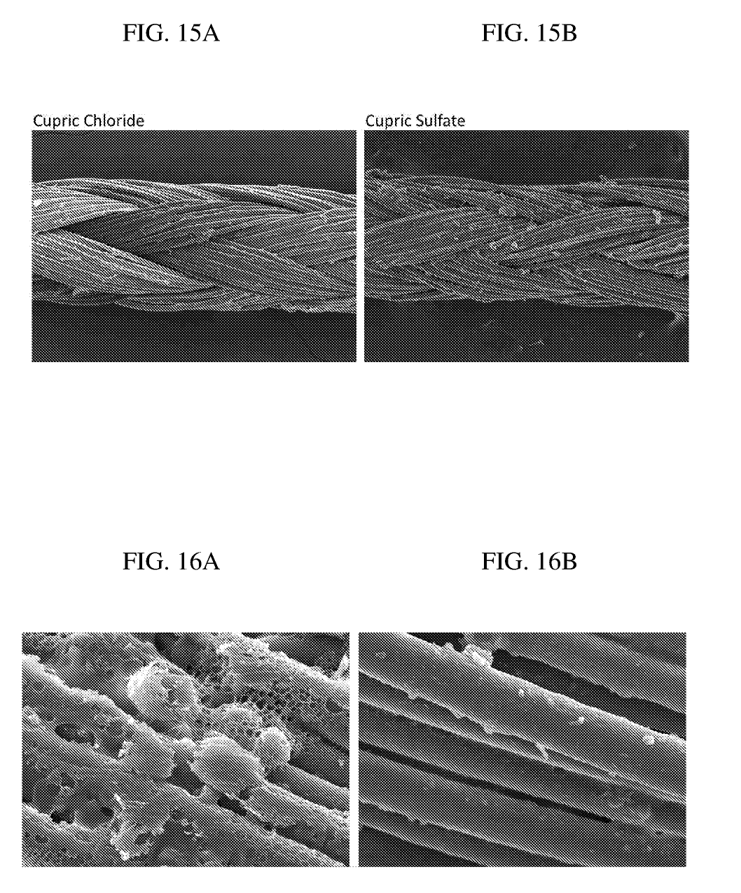

FIGS. 15A and 15B show sutures coated with copper chloride and copper sulfate, respectively. A comparison of FIGS. 15A and 15B shows that the suture with copper chloride has a relatively smooth surface while the suture with copper sulfate includes surface particles.

Coated Suture Drying Conditions:

Sutures were coated with different coating solutions as described above. After coating sutures were dried for 72 hours in RT to evaporate the solvents. Two methods of drying were tested;

Drying the sutures in an open air environment; and

Drying the sutures in a closed environment. The closed environment was provided by drying each suture in a separate container covered with aluminum foil.

As seen in FIG. 16A, drying the suture in an open air environment results in a coating surface with many surface irregularities and many cavities. In contrast, as seen in FIG. 16B, drying the suture in a controlled drying environment as described above, where the solvent evaporation rate is controlled, prevents rapid solvent evaporation and results in a relatively smooth coating surface.

ICP-OES Analysis:

The ICP-OES method is used to analyze the actual amount of copper on the sutures after the coating process. 2 types of sutures were analyzed, those prepared as described in samples 1A2 (copper chloride) and 2A2 (copper sulfate).

The suture samples were digested in 5 ml of HNO3 65% and 1 mL of HCl 37%. Digestion was carried out in quartz vessels using a “Discover” sample digestion system at high temperature and high pressure (CEM, USA). Vessels were cooled down and the volume was made up to 20 mL with deionized water. The samples were dissolved completely. Element concentration was measured in the clear solutions using an axial ICP-OES model ‘ARCOS’ from Spectro GMBH, Germany. Measurements were calibrated with standards for ICP from Merck. Element concentrations that exceeded the linear dynamic range were diluted and reanalyzed. Dilution was made using calibrated pipettes. The continuing calibration verification standard was measured to check the instrument stability.

Table 11 shows the calculated amount of copper on two types of coated sutures: suture coated with copper chloride and suture coated with copper sulfate. Calculations were based on the ICP-OES method.

TABLE 11

Copper

| Sample name | mg/kg | % wt/wt |
|---|---|---|
| Copper chloride | 1848 | 0.185 |
| Copper sulfate | 241 | 0.024 |

The copper amount in the copper chloride sample (0.185% wt/wt) is close to the calculated theoretical amount (0.266% wt/wt), which indicates on relatively high yield of the coating process with copper chloride.

As for the copper sulfate, the measured copper amount (0.024% wt/wt) is very low compare to the theoretical amount (0.207% wt/wt). The low value is supported by the SEM results as detailed above.

Copper Ion Release:

Sutures samples with copper chloride as prepared in examples 1A2 and 1A3 were analyzed for copper ion release. The test was done on two different sutures amount in the water medium: high concentration (17 cm long suture segments were immersed in 1.5 ml deionized water) and low concentration (5 cm long suture segments were immersed in 1 ml deionized water).

The tube with the suture was placed in a 37° C. shaking bath for various times: 1, 4, 24, 48, 96 and 168 hrs. At the end of each time point, a copper test strip/indicator (0-3 mg/ml) [(AquaCheck by Hach, USA) product no 2745125] was inserted into the tube for 5 sec. In the presence of copper the color of the strip changes after 1 minute. We compared the strip color to the product label in order to measure the copper amount in the medium.

At each time point the medium (deionized water) was replaced with fresh deionized water.

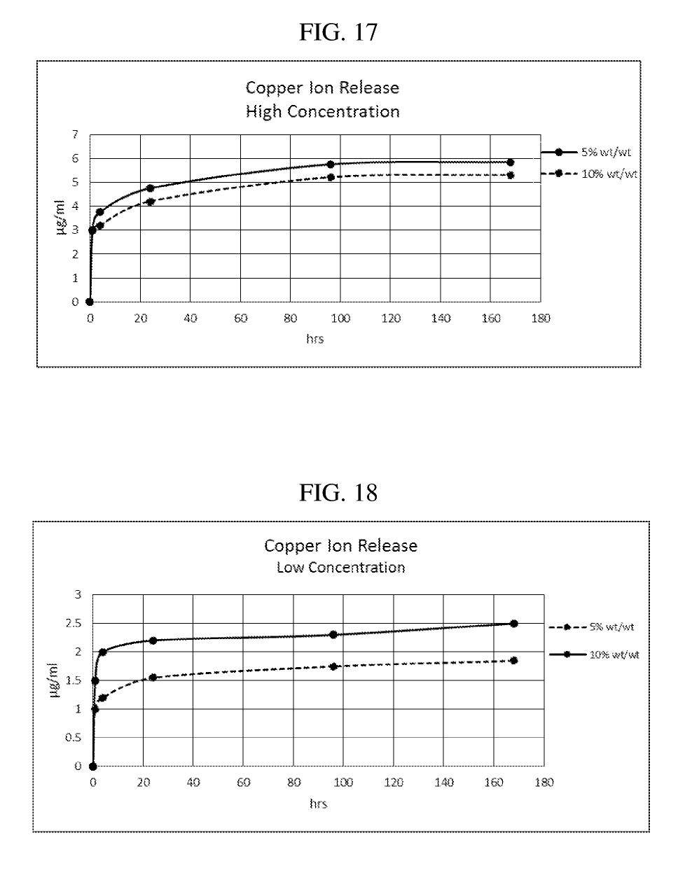

FIGS. 17 and 18 are graphs showing the copper ion release profiles for a high concentration suture and a low concentration suture, respectively.

As seen from FIGS. 17 and 18, both concentrations show a burst release of the copper ion within the first hour and a relatively steady release for up to 7 days for both concentrations.

Antibacterial Activity Assay

Assay 1

The antimicrobial efficacy of the compositions disclosed herein and their antimicrobial activity was determined by immersing copper ion containing articles or sutures, prepared as described herein, into a saline solution containing viable bacteria, including E. coli, S. aureus, Pseudomonas aeruginosa, at a defined concentration. At given time points after immersion of the article or suture into the bacterial solution, the sample was plated on nutrient agar at various dilutions in order to calculate the amount of Colony forming units (CFUs) remaining at each time point. The calculated decrease in bacterial count in the solution provides evidence of the antibacterial activity of the copper ion releasing suture.

A copper ion containing suture prepared as described hereinabove in 1A3 COPPER CHLORIDE Solution 1A—10% wt/wt was placed in lml saline solution containing 10̂5 CFUs of S. aureus. Various lengths of coated sutures, 10 cm and 15 cm, representing different concentrations, were immersed into each tube containing saline and bacteria. After 4 hours the suture segment was removed from each tube and the saline was plated on nutrient agar at various dilutions, then incubated at 37° C. for 72 hours to calculate remaining CFUs per ml.

The results show a reduction of bacterial count in solution after 4 hours incubation with copper ion containing suture:
10 cm: 99% reduction
15 cm: ≥99% reduction

Assay 2

The antibacterial efficacy of the compositions disclosed herein and their antibacterial activity were determined by using the following two methods:
Zone of inhibition assay (ZOI Assay)
In vitro colonization assay

Zone of Inhibition:

Copper ion containing sutures prepared as describes hereinabove were tested for ZOI.

Five centimeter long sections of copper ion coated sutures, prepared as described herein, were placed on bacterial lawn containing petri dishes and challenged in vitro. The Petri dishes contained S. aureus ATCC 6538 with approximately 10⁵ colony-forming units (CFU)/plate in LB agar (LBA) or Mannitol Salt agar (MSA) Petri dishes. The plates were incubated at 37° C. for 48 h and then the zone of inhibition (ZOI) was measured.

Copper ion containing sutures prepared as described hereinabove in 1A2 (calcium stearate) and 1A2 (copper stearate) were tested for ZOI and commercially coated sutures without antibacterial agent (VICRYL™ by Ethicon) were tested as a negative control. Coated VICRYL™ Suture (polyglactin 910) is a synthetic absorbable sterile surgical suture composed of a copolymer made from 90% glycolide and 10% L-lactide. Coated VICRYL™ Suture is prepared by coating Coated VICRYL™ Suture material with a mixture composed of equal parts of copolymer of glycolide and lactide (polyglactin 370) with calcium stearate.

1C2 (calcium stearate) sutures, prepared with coating solution containing 0.2% Cu with calcium stearate, results indicate a zone of inhibition of 2.37 mm and 1.92 mm (LBA and MSA respectively).

1C2 (copper stearate) sutures, prepared with coating solution containing 0.2% Cu with copper stearate results indicate a zone of inhibition of 2.74 mm and 2.16 mm (LBA and MSA respectively).

The Vicryl negative control sutures showed no zone of inhibition in both agars tested.

Table 12 summarizes the ZOI results.

TABLE 12

| | Tested | | Tested | ZOI |
|---|---|---|---|---|
| Sample name | bacteria | CFU | agar | [mm] |
| 1A2 (CaSt) | s. aureus | 10⁵ | LBA | 2.37 |
| | s. aureus | 10⁵ | MSA | 1.92 |
| 1A2 (CuSt) | s. aureus | 10⁵ | LBA | 2.74 |
| | s. aureus | 10⁵ | MSA | 2.16 |
| Coated Vicryl | s. aureus | 10⁵ | LBA | 0 |
| | s. aureus | 10⁵ | MSA | 0 |

In Vitro Colonization Assay:

0.2% Cu with calcium stearate sutures, 1C2-100 prepared as describes hereinabove were tested for in vitro colonization and compared to a commercially coated suture without antibacterial agent (Vicryl by Ethicon), as negative control. Five centimeter sections of copper ion containing sutures were placed in a solution of simulated body fluid (20% calf serum in 0.85% saline), in a dynamic model (with rotation), in sterile capped tubes. Samples then were inoculated with S. aureus ATCC 6538 at approximately 10⁵ and 10⁶ colony-forming units (CFU/tube), and incubated at 37° C. for 48 h at 100 rpm rotation. After incubation, the suture samples were removed, and washed with saline to remove free S. aureus. Bacteria that colonized the suture surfaces of the suture were collected by sonification of the suture in saline solution for 5 minutes, followed by serial dilutions and drop plating on tryptic soy agar (TSA) plates to calculate the number of bacteria per ml and per suture section. The plates were incubated at 37° C. for 24 h, and subsequent bacterial counts (30-300) was performed and reported as CFU/suture and Log 10 CFU/suture.

The results indicated a 1 log reduction in growth response in the sutures which were coated with copper compounds and no reduction for the negative control samples.

Table 13 summarizes the in-vitro colonization results.

TABLE 13

| Sample name | Tested bacteria | CFU | Log reduction |
|---|---|---|---|
| 1A2 (CaSt) | s. aureus | 10⁶ | 1 |
| Coated Vicryl | s. aureus | 10⁶ | 0 |

The following examples exemplify additional processes for preparing coating solutions containing varying amounts of copper chloride as follows and as per the following Table 14:

Variable parameters of the coating solution for the coating procedure include various concentrations of the co-polymer/stearate components as well as the copper compound concentrations. These variable concentrations of the different components from the total coating solution (% wt/wt) are exemplified in the following examples as summarized in Table 14. Table 14 shows the compositions of the various coating solutions indicating the amounts of Co-polymer/stearate and Copper compound in each solution

TABLE 14

| Co-polymer/stearate | Copper compound |
|---|---|
| [% wt/wt] | [% wt/wt] |
| 2 | 1.25 |
| | 2.5 |
| | 4.375 |
| | 6.25 |
| 5 | 1.25 |
| | 2.5 |
| | 4.375 |
| | 6.25 |
| 10 | 1.25 |
| | 2.5 |
| | 4.375 |
| | 6.25 |

2% Wt/Wt Coating Solutions

General Formula for Preparation of 2% Wt/Wt Co-Polymer/Stearate Coating Solutions (Containing 1.25-6.25% CuCl₂ wt/wt in Solution):

40 g of the copolymer 65:35 P(D,L)LGA, 40 g of calcium stearate or copper stearate and the desired amount of copper chloride (CuCl₂) was added to a 5 liter closed glass bottle containing MEK solvent to a final solution weight of 4000 gr. All of the ingredients were mixed together using a magnetic stirrer overnight at room temperature producing a uniform suspension in which small particles were visible. Examples of various CuCl₂ concentration solutions were prepared using 50 gr, 100 gr, 175 gr and 250 gr, resulting in coated sutures described below 1C1-50, 1C1-100, 1C1-175 and 1C1-250 respectively.

1C1-100-2% Wt/Wt Co-Polymer/Stearate Coating Solution Containing (w/0.2% Cu):

40 g of the copolymer 65:35 P(D,L)LGA, 40 g of calcium stearate or copper stearate and 100 g of copper chloride (CuC12) were added to 3820 g of MEK in a 5 liter closed glass bottle. All the ingredients were mixed together using a magnetic stirrer overnight at room temperature producing a uniform suspension in which small particles were visible.

5% Wt/Wt Coating Solutions

General Formula for Preparation of 5% Wt/Wt Co-Polymer/Stearate Coating Solutions (Containing 1.25-6.25% CuCl₂ wt/wt in Solution):

100 g of the copolymer 65:35 P(D,L)LGA, 100 g of calcium stearate or copper stearate and the desired amount of copper chloride (CuCl₂) was added to a 5 liter closed glass bottle containing MEK solvent. MEK was added to a final solution weight of 4000 gr. All the ingredients were mixed together using a magnetic stirrer overnight at room temperature producing a uniform suspension in which small particles were visible. Examples of various CuCl₂ concentration solutions were prepared using 50 gr, 100 gr, 175 gr and 250 gr were prepared, resulting in coated sutures described below 1C2-50,1C2-100, 1C2-175 and 1C2-250 respectively.

1C2-100—5% Wt/Wt Co-Polymer/Stearate Coating Solution (w/0.2% Cu):

100 g of the copolymer 65:35 P(D,L)LGA, 100 g of calcium stearate or copper stearate and 100 g of copper chloride (CuCl₂) were added to 3700 g of MEK in a 5 liter closed glass bottle. All the ingredients were mixed together using a magnetic stirrer overnight at room temperature producing a uniform suspension in which small particles were visible.

10% Wt/Wt Coating Solutions

General Formula for Preparation of 10% Wt/Wt Co-Polymer/Stearate Coating Solutions (Containing 1.25-6.25% CuCl₂ wt/wt in Solution):

200 g of the copolymer 65:35 P(D,L)LGA, 200 g of calcium stearate or copper stearate and the desired amount of copper chloride (CuCl₂) was added to a 5 liter closed glass bottle containing MEK solvent. MEK was added to a final solution weight of 4000 gr. All the ingredients were mixed together using a magnetic stirrer overnight at room temperature producing a uniform suspension in which small particles were visible. Examples of various CuCl₂ concentration solutions were prepared using 50 gr, 100 gr, 175 gr and 250 gr were prepared, resulting in coated sutures described below 1C3-50, 1C3-100, 1C3-175 and 1C3-250 respectively.

1C3-100-10% Wt/Wt Co-Polymer/Stearate Coating Solution (w/0.2% Cu):

200 g of the copolymer 65:35 P(D,L)LGA, 200 g of calcium stearate or copper stearate and 100 g of copper chloride (CuCl₂) were added to 3500 g of MEK in a 5 liter closed glass bottle. All the ingredients were mixed together using a magnetic stirrer overnight at room temperature producing a uniform suspension in which small particles were visible.

Table 15 summarizes the components of the different coating solutions prepared as described in 1C above for copper chloride in different coating solutions containing 2%, 5% and 10% wt/wt co-polymer/stearate coating solution, 1C1, 1C2, 1C3, respectively, where the % represents the total amount of solids in solution including 65:35 P(D,L)LGA copolymer and calcium stearate or copper stearate as the case may be including the copper compound variable concentrations in the solution.

TABLE 15

| Coating | 65:35 | Calcium | Copper | Cupric | |
|---|---|---|---|---|---|
| solution | PDLLGA | stearate | stearate | chloride | EMK |
| [% wt/wt] | [g] | [g] | [g] | [g] | [g] |
| 2% | 40 | 40 | — | 50 | 3870 |
| | 40 | 40 | — | 100 | 3820 |
| | 40 | 40 | — | 175 | 3745 |
| | 40 | 40 | — | 250 | 3670 |
| | 40 | — | 40 | 50 | 3870 |
| | 40 | — | 40 | 100 | 3820 |
| | 40 | — | 40 | 175 | 3745 |
| | 40 | — | 40 | 250 | 3670 |
| 5% | 100 | 100 | — | 50 | 3750 |
| | 100 | 100 | — | 100 | 3700 |
| | 100 | 100 | — | 175 | 3625 |
| | 100 | 100 | — | 250 | 3550 |
| | 100 | — | 100 | 50 | 3750 |
| | 100 | — | 100 | 100 | 3700 |
| | 100 | — | 100 | 175 | 3625 |
| | 100 | — | 100 | 250 | 3550 |
| 10% | 200 | 200 | — | 50 | 3750 |
| | 200 | 200 | — | 100 | 3500 |
| | 200 | 200 | — | 175 | 3425 |
| | 200 | 200 | — | 250 | 3350 |
| | 200 | — | 200 | 50 | 3750 |
| | 200 | — | 200 | 100 | 3500 |
| | 200 | — | 200 | 175 | 3425 |
| | 200 | — | 200 | 250 | 3350 |

### Example 23 Coating a Suture with Coating Solution

The Coating Solution as described in 1C above was circulated in a closed bath system (coating bath). Uncoated sutures, polyglactin 910 sutures, copolymers made from 90% glycolide and 10% L-lactide, are drawn through the coating bath at an appropriate draw speed in order to obtain the desired amount of coating on the suture whereas slower draw speeds results in higher coating per suture segment and faster draw speeds results in lower coating concentrations per suture segment. For example 1, 5, 10, 15, 20, 25, 30 or 40 meter/min may be used. Each section of the suture is passed through the bath at least once, however each section may be passed twice or three times or more through the bath until a desired coating is achieved. After passing through the coating bath and when a desired coating is achieved, the coated sutures are then passed through a tubular heating channel at an appropriate rate to optimize the drying, complete or nearly complete evaporation of the solvent and produce an optimal coating surface on the suture. For example the coated suture may be passed through the heating channel at the same rate as the coating bath or at a different rate, slower or faster than the rate of the coating bath. For example it may be passed at a rate ofl, 5, 10, 15, 20, 25, 30 or 40 meter/min. The drying temperature may range from 120° C. to 210° C. For example the drying temperature is optimally set at 175° C. Residual amounts of solvent may be further evaporated at room temperature in an appropriate container which may be an open container.

### Example 24 Coating a Suture with 1C2-100 Coating Solution 5% Wt/Wt Co-Polymer/Stearate Coating Solution (w/0.2% Cu

Coating Procedure:

5% wt/wt coating solution with 100 g CuCl₂ is circulated in a closed bath system. The uncoated sutures, polyglactin 910 sutures, composed of copolymer made from 90% glycolide and 10% L-lactide, were drawn through the coating bath and coated at a speed of 10 meter/min. Each section of the required suture length was passed through the bath once.

Solvent Evaporation and Annealing:

After passing through the coating bath, the coated sutures continuously entered into a 1.5 m tubular heating channel at a rate of 10 meter/min and set at 175° C., for drying. Before packaging the sutures were dried at 50° C. for 24 hours to reach a water content below 500 ppm.

Sutures were weighed before (W1) and after (W2) they were coated with the coating solution. The coating weight was calculated by the following equation:

% coating=(W2−W1)/W1*100%

The coating weight include the copolymer, stearate and copper chloride components (the solvent is evaporate in the dry process).

The percentage of the CuCl₂ was calculated from the total coating weight and was multiple with 47.26% (the copper percentage from the CuCl₂ compound).

The value of the copper was divided by the suture weight after coating to receive the copper wt out of the suture wt.

### General Example 25 Coating a Suture with 1C1 Coating Solution: 2, 5 or 10% Wt/Wt Co-Polymer/Stearate Coating Solution (w/0.1-0.5% Cu)

2, 5% or 10 wt/wt coating solution with 50, 100, 175 or 250 g CuCl₂ was circulated in a closed bath system. The uncoated sutures, polyglactin 910 sutures, composed of copolymer made from 90% glycolide and 10% L-lactide, were drawn through the coating bath and coated at a speed of 10 meter/min. Each section of the required suture length was passed through the bath once. After passing through the coating bath, the coated sutures continuously entered into a 1.5 m tubular heating channel at a rate of 10 meter/min and set at 175° C., for drying. Before packaging the sutures were dried at 50° C. for 24 hours to reach a water content below 500 ppm.

Using these specific coating conditions the estimated copper amount on the sutures (% wt/wt) at the end of the coating process are exemplified in the following table 16:

TABLE 16

| | | | Estimated Cu |
|---|---|---|---|
| Co-polymer/ | | | content on the |
| stearate | Sample | Copper compound | suture |
| [% wt/wt] | name | [% wt/wt] | [% wt/wt] |
| 2 | A1 | 1.25 | 0.1 |
| | A2 | 2.5 | 0.2 |
| | A3 | 4.375 | 0.35 |
| | A4 | 6.25 | 0.5 |
| 5 | B1 | 1.25 | 0.1 |
| | B2 | 2.5 | 0.2 |
| | B3 | 4.375 | 0.35 |
| | B4 | 6.25 | 0.5 |
| 10 | C1 | 1.25 | 0.1 |
| | C2 | 2.5 | 0.2 |
| | C3 | 4.375 | 0.35 |
| | C4 | 6.25 | 0.5 |

Analysis of the Sutures:

ICP-OES Analysis:

The ICP-OES method as described above is used to analyze the actual amount of copper coated onto the sutures during the coating and evaporation process. For example, 2 types of sutures were analyzed by ICP, those prepared as described in samples 1C2-100 with calcium stearate and 1C2*-100 with copper stearate.

Table 17 shows the calculated amount of copper on two types of coated sutures: suture coated with copper chloride and calcium stearate and suture coated with copper chloride and copper stearate. Calculations were based on the ICP-OES method.

TABLE 17

| | | | | | | ICP |
|---|---|---|---|---|---|---|
| | Coating solution | | | | | Copper |
| Sample | PDLLGA | CaSt | CuSt | CuCl₂ | | (average |
| name | 65:35 [g] | [g] | [g] | [g] | Solvent | value) [ppm] |
| 1C2-100 | 100 | 100 | 0 | 100 | MEK | 1977 |
| 1C2*- | 100 | 0 | 100 | 100 | MEK | 2372 |
| 100 | | | | | | |

*Sutures with Copper stearate instead of calcium stearate.

The copper amount measured (sample 1C2-100) in the copper chloride sample with calcium stearate is 0.1977% wt/wt is close to the calculated theoretical amount (approximately 0.2% wt/wt), indicating high deposition yield during the coating process using copper chloride.

The copper amount measured (sample 1C2*-100) in the copper chloride sample with copper stearate is 0.2372% wt/wt is similar to the calculated theoretical amount (0.2423% wt/wt), indicating a high deposition yield during the coating process using copper chloride.

Copper Ion Release:

Sutures samples with copper chloride as prepared in examples 1C2-100 with calcium stearate and 1C2*-100 with copper stearate were analyzed for copper ion release at various time points.

For each time point a 5 meter suture was placed in a 50 ml PP-vessel with exactly 50 ml of double distilled water, in a shaking bath at 37° C.±2° C. The following time points were tested: 1, 4, 24, 48, 96 and 168 hrs. At the end of each time point, the suture was removed from the tube and the total 50 ml volume was tested for copper content by ICP, as described above.

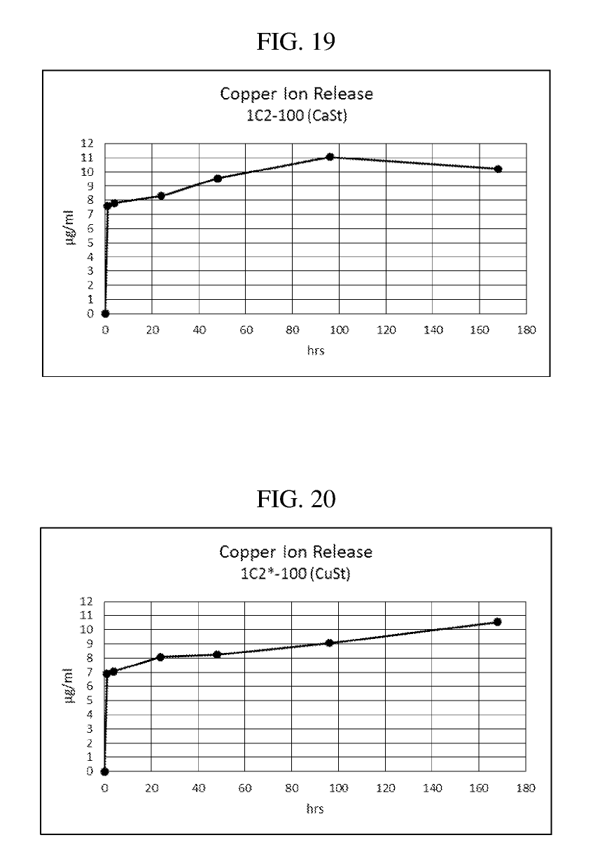

FIGS. 19 and 20 are graphs showing the copper ion release profiles for 1C2-100 (calcium stearate) and 1C2*-100 (copper stearate), respectively.

As seen from FIGS. 19 and 20, both concentrations show a burst release of the copper ion within the first hour followed by an additional steady gradual release for up to 7 days, for both suture coating preparations.

### Example 26 Surgical Degradable Mesh

General Description:

This embodiment of the invention relates to coating synthetic, bioabsorbable polymer materials and implants, like fibers, sutures, meshes and other tissue management, wound closure and tissue engineering devices with an antimicrobial agent. The invention also relates to methods of preventing and treating infections by applying the coating using synthetic, bioabsorbable aliphatic polyesters, including poly(ε-caprolactone) (PCL), polylactide (PLA), polyglycolide (PGA), polydioxanone (PDO), or copolymers thereof.

Antimicrobial Coating

The antimicrobial agent, such as copper, is applied to mesh in a coating solution containing dissolved copper ions, a biodegradable polymer and various additives. The copper ions include: copper chloride (CuCl₂) and copper sulfate (CuSO₄).

Mesh prostheses are used in hernia repair and in other surgical procedures, including the repair of anatomical defects of the abdominal wall, diaphragm, and body walls, correction of defects in the genitourinary system, and repair of traumatically damaged organs such as the spleen, liver or kidney or in inducing the formation of fibrous tissue small joint in fingers of rheumatoid patients (U.S. Pat. No. 6,113,640) or as scaffolds for tissue engineering (Gaissmaer et al. 2002, Lansman et al. 2002).

The multifunctional devices of the present invention can be made in any appropriate form to contain a polymer matrix and antibiotic(s), employing polymer technological processing methods. Typical forms are mono- and/or multifilamentous sutures and their derivatives such as meshes and scaffolds.

Absorbable Mesh Coated with Antimicrobial Agent:

Copper Chloride

In a specific example a copolymer of PLGA with 90% Glycolide (G) and 10% L-Lactide (L).

The copper compounds are dissolved in a solution of P(D,L)LGA (65% D,L-Lactide and 35% Glycolide) and calcium stearate or copper stearate, as follows:

Solution I:

65:35 P(D,L)LGA is dissolved in ethyl acetate or acetone in a glass container covered with aluminum foil. The copolymer solution is mixed using a magnetic stirrer at room temperature (RT) until all compounds are fully dissolved forming a clear solution. Calcium stearate or copper stearate are added to solution and are mixed for an additional 1 hour.

Solution II:

Copper chloride (CuCl₂) is dissolved in acetone and magnetically stirred in a separate flask for 30 min at RT until clear solution.

Then, solutions 1 and 2 are mixed together for additional 15 min. at RT, covered with aluminum foil.

The mesh is dipped in the coating solution for few seconds (5, 10 or 15 seconds) and is dried in a sealed compartment until full solvent evaporation.

The coating loading is varied within the range of 2-10% wt./wt. coating over the mesh.

Copper Sulfate

In a specific example a copolymer of PLGA with 90% Glycolide (G) and 10% L-Lactide (L).

The copper compounds are dissolved in a solution of P(D,L)LGA (65% D,L-Lactide and 35% Glycolide) and calcium stearate or copper stearate, as follows:

65:35 P(D,L)LGA is dissolved in ethyl acetate or in acetone in a glass container sealed with aluminum foil. The copolymer solution is mixed using a magnetic stirrer at RT until all compounds are fully dissolved and a clear solution is formed. Calcium stearate or copper stearate are added to solution and are mixed for an additional 1 hr.

Copper sulfate (CuSO₄) is added to the solution and is mixed for additional 1.5 hrs.

The mesh is dipped coated for few seconds (5, 10 or 15 seconds) and is dried in a sealed compartment until full solvent evaporation.

The coating loading is varied within the range of 2-10% wt./wt. coating over the mesh.

### Example 27—Antimicrobial Suture Needles

General Description:

Surgical needles and attached surgical sutures are used in most surgical procedures for a variety of applications including tissue repair and approximation and securing medical devices to tissue including mesh implants to support organs, vascular grafts to connect to blood vessels or even artificial heart valves.

Surgical needles piercing the tissue play a key role in spreading infections and viruses into the treated site. Therefore, it is necessary to have adequate measures to prevent the occurrence of such contaminations.

Herein are described methods for coating suture needles with copper ions as an antibacterial agent employing different coating techniques described below including dip-coating in a polymeric solution containing the pre-dissolved copper ions.

Coating Surgical Needles with Antibacterial Solutions Dip Coating with P(D,L)LGA and Copper Chloride Solutions

Surgical needles made of stainless steel attached to surgical sutures (absorbable or non-absorbable suture) are coated by clip coating within an organic solution of biodegradable polymer pre-dissolved with antibacterial copper ions as describe in detail the following:

Solution I:

65:35 P(D,L)LGA is dissolved in ethyl acetate or acetone in a glass container covered with aluminum foil to control evaporation. The copolymer solution is mixed using a magnetic stirrer at room temperature (RT) until all compounds are fully dissolved forming a clear solution. Calcium stearate or copper stearate are added to solution and mixed for an additional 1 hour.

Solution II:

Copper chloride (CuCl₂) is dissolved in acetone and magnetically stirred in a separate flask for 30 min at RT until clear solution.

Solutions I and II are then mixed together for additional 15 minutes at RT, covered with aluminum foil.

The needle is dipped in the coating solution for a brief period of 5, 10 or 15 seconds and then is dried in a sealed compartment until solvent is fully evaporated.

The coating load varies between a range of 2-10% wt/wt coating/total needle weight.

Dip Coating with P(D,L)LGA and Copper Sulfate Solutions

65:35 P(D,L)LGA is dissolved in ethyl acetate or in acetone in a glass container sealed with aluminum foil to control evaporation. The copolymer solution is mixed using a magnetic stirrer at RT until all compounds are fully dissolved and a clear solution is formed. Calcium stearate or copper stearate are added to solution and mixed for an additional 1 hr.

Copper sulfate (CuSO₄) is added to the solution and is mixed for additional 1.5 hrs.

The needle is dipped in the coating solution for a brief period of 5, 10 or 15 seconds and then is dried in a sealed compartment until solvent is fully evaporated.

The coating load varies between a range of 2-10% wt/wt coating/total needle weight

### Example 28 Antimicrobial Wound Dressing

General Description:

A wound dressing comprising a flexible base layer and an antimicrobial material, wherein the antimicrobial material comprises copper ions, a carrier of biodegradable polymer and other additives. The copper ions include: copper chloride (CuCl₂) and copper sulfate (CuSO₄).

This dressing can optionally comprise a pressure sensitive adhesive component and other therapeutically active components. Those components can be either incorporated into the antimicrobial material or as a separate layer.

Antimicrobial Copper Containing Wound Dressing

In a specific example, a copolymer of PLGA with 90% Glycolide (G) and 10% L-Lactide (L), is used to coat inner and/or outer wound dressing layers, for direct skin contact or indirect skin contact. The polymer solution containing dissolved copper chloride or copper sulfate is sprayed on the wound dressing cloth or bandage. The polymer solution containing the dissolved copper chloride or copper sulfate can be also used for dip coating of the bandage cloth in the solution. Subsequently, the cloth is squeezed to remove unbounded solids. Next the cloth bandages are left to evaporate all residual solvents.

Antimicrobial Copper Chloride Containing Wound Dressing.

The copper compounds are dissolved in P(D,L)LGA (65% D,L-Lactide and 35% Glycolide) and calcium stearate or copper stearate solution, as follows:

Solution I:

65:35 P(D,L)LGA copolymer is dissolved in ethyl acetate or acetone in a glass container covered with aluminum foil. The copolymer solution is mixed using a magnetic stirrer at room temperature (RT) until all compounds are fully dissolved forming a clear solution. Calcium stearate or copper stearate are added to solution and mixed for an additional 1 hour.

Solution II:

Copper chloride (CuCl₂) is dissolved in acetone and is magnetically stirred in a separate flask for 30 min at RT until clear solution.

Solutions I and II are then mixed together for additional 15 minutes at RT, covered with aluminum foil to prepare a combined solution. The bandage is spray coated with the combined solution for a few seconds, typically 5, 10 or 15 seconds, until fully coated and is dried in a sealed compartment until complete evaporation of solvents.

The coating load on bandage varies between the range of 5-15% wt/wt coating per bandage weight.

Antimicrobial Copper Sulfate Containing Wound Dressing.

The copper sulfate is dissolved in a solution of P(D,L)LGA (65% D,L-Lactide and 35% Glycolide) and calcium stearate or copper stearate, as follows:

Solution I:

65:35 P(D,L)LGA is dissolved in ethyl acetate or acetone in a glass container covered with aluminum foil. The copolymer solution is mixed using a magnetic stirrer at room temperature (RT) until all compounds are fully dissolved forming a clear solution. Calcium stearate or copper stearate are added to solution and mixed for an additional 1 hour.

Solution II:

Copper Sulfate (CuSO₄) is dissolved in acetone and is magnetically stirred in a separate flask for 30 min at RT until clear solution.

Solutions I and II are then mixed together for additional 15 minutes at RT, covered with aluminum foil to prepare a combined solution. The bandage is spray coated with the combined solution for a few seconds (5, 10 or 15 seconds) until fully coated and is dried in a sealed compartment until complete evaporation of solvents.

The coating load on bandage varies between the range of 5-15% wt/wt coating per bandage weight.

As noted above, the methods of embedding copper ions described above and the method of coating described hereinabove may be combined into a single product. Thus, the coating methods described in examples 22-25 above may also be utilized to coat any of the monofilaments, multifilament and sutures prepared utilizing any of examples 1-3, 7 and 9-17 described hereinabove. Additionally, the coating method described in example 26 above may be utilized to coat any of the meshes prepared utilizing any of examples 18-19 described hereinabove.

It will be appreciated by persons skilled in the art that the present invention is not limited by what has been particularly shown and described hereinabove. Rather the scope of the present invention includes both combinations and subcombinations of features described hereinabove and variations and modifications thereof which are not in the prior art.

1. A suture comprising:
at least one filament formed of at least one polymer; and
a biodegradable coating, comprising copper chloride, including at least one copper ion at least partially coated on said at least one filament in a manner such that said at least one copper ion is released from said biodegradable coating over time.

2. (canceled)

3. A suture according to claim 1 and wherein said biodegradable coating comprises at least one biodegradable polymer.

4. A suture according to claim 1 and wherein said biodegradable coating comprises at least one aliphatic polyester.

5. A suture according to claim 4 and wherein said at least one aliphatic polyester is selected from the group consisting of: polymers polymerized from one or more of the following: ε-caprolactone, lactide, glycolide, dioxanone and copolymers thereof.

6. A suture according to claim 4 and wherein said at least one aliphatic polyester comprises PLGA.

7. A suture according to claim 1 and wherein said biodegradable coating comprises a copolymer made from 65% D,L-lactide and 35% glycolide.

8-19. (canceled)

20. A suture according to claim 1 and where said biodegradable coating also comprises at least one lubricant.

21. A suture according to claim 20 and wherein said at least one lubricant is selected from the group consisting of copper stearate and calcium stearate.

22. A suture according to claim 1 and wherein said at least one filament is biodegradable.

23-25. (canceled)

26. A biocompatible article comprising:
at least one element formed of at least one polymer; and
a biodegradable coating, comprising copper chloride, including at least one copper ion at least partially coated on said at least one element in a manner such that said at least one copper ion is released from said biodegradable coating over time.

27. (canceled)

28. A biocompatible article according to claim 26 and wherein said biodegradable coating comprises at least one biodegradable polymer.

29-31. (canceled)

32. A biocompatible article according to claim 26 and wherein said biodegradable coating comprises a copolymer made from 65% D,L-lactide and 35% glycolide.

33-44. (canceled)

45. A biocompatible article according to claim 26 and wherein said at least one element is biodegradable.

46. A biocompatible article according to claim 26 and where said biodegradable coating also comprises at least one lubricant.

47-108. (canceled)

109. A method of manufacture of a suture, the method comprising:
providing at least one filament comprising at least one polymer; and
at least partially coating said at least one filament with a biodegradable coating, comprising copper chloride, including at least one copper ion in a manner such that said at least one copper ion is released from said biodegradable coating over time.

110. (canceled)

111. A method according to claim 109 and also comprising adding at least one lubricant to at least one of said at least one filament and said biodegradable coating.

112. A method of manufacture of a biocompatible article, the method comprising:
providing at least one element comprising at least one polymer; and
at least partially coating said at least one element with a biodegradable coating, comprising copper chloride, including at least one copper ion in a manner such that said at least one copper ion is released from said biodegradable coating over time.

113. (canceled)

114. A method according to claim 112 and also comprising adding at least one lubricant to said biodegradable coating.

115-140. (canceled)

141. A method according to claim 112 and wherein said biodegradable coating comprises at least one biodegradable polymer.

142. A method according to claim 112 and wherein said biodegradable coating comprises a copolymer made from 65% D,L-lactide and 35% glycolide.

143. A method according to claim 112 and wherein said at least one element is biodegradable.
